(19) United States
(12) Patent Application Publication
(10) Pub. No.: US 2026/0218112 A1
(43) Pub. Date: Jul. 30, 2026

(54) PRESSURIZED AIR-ASSISTED CELL DISPENSING SYSTEM

(71) Applicant: Astrin Biosciences, Inc., St. Paul, MN (US)

(72) Inventors: Mahdi AHMADI, Minneapolis, MN (US); Jayant PARTHASARATHY, Excelsior, MN (US); Jiarong HONG, Eden Prairie, MN (US)

(73) Assignee: Astrin Biosciences, Inc., St. Paul, MN (US)

(21) Appl. No.: 19/152,628

(22) PCT Filed: Feb. 2, 2024
(86) PCT No.: PCT/US2024/014344

§ 371 Date: Jul. 31, 2025

Related U.S. Application Data
(60) Provisional application No. 63/482,917, filed on Feb. 2, 2023.

(51) Int. Cl. C12M 1/00; C12M 1/26

(52) U.S. Cl. CPC C12M 47/04; C12M 33/04

(57) ABSTRACT

The present embodiments relate to a pressurized air-assisted dispenser. The present embodiments generally can provide a microfluidic approach to achieve high precision single cell dispensing for various omic analyses. Omics includes, for example, proteomics, transcriptomics, genomics, metabolomics, lipidomics, and epigenomics, which correspond to global analyses of proteins, RNA, genes, metabolites, lipids, and methylated DNA or modified histone proteins in chromosomes, respectively. Particularly, in comparison to other single particle and cell dispensing systems, the present devices and methods introduce compressed air to reduce the dispensing droplet size and control the uniformity and timing of the droplet generation and dispensing. Furthermore, imaging methods can be used to determine the droplets with targeted particles or cells and assist the dispense of the droplets into the selected particle or cell collecting containers.

### PRIORITY

This application claims the benefit of U.S. Ser. No. 63/482,917, filed on Feb. 2, 2023, which is incorporated by reference herein in its entirety.

### TECHNICAL FIELD

The methods and compositions described here relate to the field of single particle or cell isolation systems. More particularly, embodiments relate to a pressurized air-assisted cell dispensing system using one or more air channels to direct pressurized air to release a droplet containing a target particle or cell from a dispenser.

### BACKGROUND

Single cell isolation can include separating cells (and other particles of interest) in a biological fluid and isolating individual cells for further processing. Single cell isolation can be used a prerequisite for clonal cell line development and for effective clonal cultivation and omic profiling at single cell level.

In many cases, single cell isolation techniques can include limiting dilution, fluorescence-activated cell sorting (FACS), single-cell dispensing (SCD), various microfluidic single cell isolation methods using hydrodynamic trapping, nanowells, and droplet microfluidics, as well as single-cell manipulation using optical tweezers, dielectrophoresis, and automated micromanipulators, for example. Among all such techniques, FACS and SCD can allow for individual cells to be enclosed inside a droplet that contains reagents with a desired volume and can further dispense the droplets into standard well plates (e.g., 96 well plates), which can be particularly suitable for downstream cell culture and single cell omic profiling.

When comparing FACS and SCD, FACS can allow for high throughput cell sorting by producing small droplets (~nL) at very fast rate. However, the fast flow rate employed in FACS can yield large numbers of empty droplets (droplets with no cells) and non-negligible impact on the cell viability, which can be detrimental to downstream single cell analysis. In contrast to continuous droplet generation in FACS, SCD can operate at much lower flow rate and can only generate and dispense droplets that contain targeted cells into the target substrate (e.g., microtiter well plate). Therefore, SCD can achieve more effective delivery of individual viable cells for single cell analysis, but it can operate at a much lower throughput and can produce the droplets in ~μL range, considerably larger those from FACS.

More efficient methods of targeted single cell isolation are needed in the art.

### SUMMARY

An aspect provides a pressurized air-assisted dispenser comprising a channel comprising a first end and a second end, the first end comprising an inlet to obtain a fluid containing particles or cells, and the second end comprising an outlet configured to form a droplet comprising a single target particle or cell and output the droplet; and at least one air channel disposed along a portion of the pressurized air-assisted dispenser adjacent to the channel, wherein the at least one air channel comprises an air channel inlet configured to obtain a flow of pressurized air and at least one air channel outlet adjacent to the outlet of the channel, wherein the output of the flow of pressurized air at the air channel outlet can be configured to cause a breakup and removal of the droplet from the outlet of the channel. The can comprise channel comprises first diameter that is tapered to a second diameter at the outlet, wherein the first diameter is larger than the second diameter. The at least one air channel can comprise two symmetrical channels disposed on multiple sides of the channel, and wherein each of the two symmetrical channels comprise an outlet that are disposed adjacent to the outlet of the channel. The dispenser can further comprise at least one sheath inlet channel, the sheath inlet channel comprising an inlet configured to obtain a portion or portions of the fluid and an outlet outputting the portion of the fluid into the channel, wherein the at least one sheath inlet channels is configured to focus the fluid containing the particle or cell to a specified width within the channel, and wherein fluid in the sheath inlet channel include one or more molecular barcodes for tagging the particle or cell. Fluid in the sheath inlet channel can include one or more molecular barcodes for tagging the particle or cell. The at least one sheath inlet channel can comprise two sheath inlet channels, wherein the specified width within the channel of the fluid containing the particles or cells in the channel is controlled by adjusting a flow rate of the portions of fluid flowing in each of the at least two sheath inlet channels.

The channel can be part of a particle or cell focusing chip, and the at least one air channel can be part of a pneumatic chip, where the particle or cell focusing chip can be disposed within the pneumatic chip. The dispenser can further comprise at least one sheath inlet disposed in the particle or cell focusing chip. The dispenser of can further comprising an imaging system comprising a light source configured to emit a light into the channel to highlight a holographic pattern and/or a fluorescent pattern of the particle or cell in the channel; and at least one image sensor configured to capture an image of the holographic pattern and/or the fluorescent pattern of the particle or cell. The dispenser can further comprise a computing node including a processor and a memory, the memory including instructions that, when executed by the processor, cause the processor to: obtain the image of the holographic pattern and/or the fluorescent pattern of the particle or cell; process the image to determine a particle or cell type based on the holographic pattern and/or fluorescent pattern of the particle or cell depicted in the image; and responsive to determining that the particle or cell type is a target particle or cell type, causing an air compressor connected to the air channel inlet of the at least one air channel to direct the flow of pressurized air to the air channel outlet to cause the breakup and removal of the droplet from the outlet of the channel, wherein the breakup and removal of the droplet is coordinated with movement of a sample collector or a waste collector moved via a translation stage.

Another aspect provides a method comprising obtaining, at an inlet of a channel of a pressurized air-assisted dispenser, a fluid containing at least one particle or cell, wherein the fluid containing the at least one particle or cell is configured to travel along the channel to an outlet; emitting, by a laser light source, a laser light into the channel to highlight a holographic pattern and/or a fluorescent pattern of the particle or cell in the channel; capturing, by an image sensor, an image of the holographic pattern and/or the fluorescent pattern of the particle or cell; processing, by a computing node, the image to determine a particle or cell type based on the holographic pattern of the particle or cell depicted in the image; and responsive to determining that the particle or cell type is a target particle or cell type, causing an air compressor connected to an air channel inlet of at least one air channel in the pressurized air-assisted dispenser to direct a flow of pressurized air to the air channel outlet disposed adjacent to the outlet of the channel to cause breakup and removal of a droplet including the target particle or cell from the outlet of the channel. The channel comprises first diameter that is tapered to a second diameter at the outlet, wherein the first diameter is larger than the second diameter. The at least one air channel can comprise two symmetrical air channels disposed on multiple sides of the channel, and wherein each of the two symmetrical channels comprise an outlet that are disposed adjacent to the outlet of the channel. The method can further comprise directing to at least two sheath inlet channels, a portion of the fluid to focus the fluid containing the particle or cell to a specified width within the channel, wherein each sheath inlet channel comprises an inlet configured to obtain a portion of the fluid and an outlet outputting the portion of the fluid into the channel. The method of can further comprise controlling the specified width within the channel of the fluid by adjusting a flow rate of the portions of fluid flowing in each of the at least two sheath inlet channels. The channel can be part of a particle or cell focusing chip, and the at least one air channel can be part of a pneumatic chip, wherein the particle or cell focusing chip is disposed within the pneumatic chip, and wherein a sheath inlet is disposed in the particle or cell focusing chip. The fluid containing at least one particle or cell can include oil droplets encapsulating the at least one particle or cell.

An aspect provides a system comprising a single particle or cell dispenser that includes a channel comprising a first end and a second end, the first end comprising an inlet to obtain a fluid containing target particle or cells, and the second end comprising an outlet configured to form a droplet comprising a single target particle or cell and output the droplet; and two air channels disposed along a portion of the single particle or cell dispenser adjacent to the channel, wherein the two air channels are configured to obtain a flow of pressurized air at an air channel inlet common to the two air channels and output the flow of pressurized air an air channel outlet for each of the two air channels, wherein the output of the flow of pressurized air at each air channel outlet is configured to cause a breakup and removal of the droplet from the outlet of the channel. The system can comprise an imaging system comprising a laser light source configured to emit a laser light into the channel to highlight a holographic and/or fluorescent pattern of the particle or cells in the channel; and at least one image sensor configured to capture an image of the holographic and/or fluorescent pattern of the particle or cells. The single particle or cell dispenser can further comprise at least two sheath inlet channels, with each sheath inlet channel comprising an inlet configured to obtain a portion of the fluid and an outlet outputting the portion of the fluid in the channel, wherein the at least two sheath inlet channels are configured to focus the fluid containing the particle or cell to a specified width within the channel, wherein the specified width within the channel of the fluid containing the particle or cell in the channel is controlled by adjusting a flow rate of the portions of fluid flowing in each of the at least two sheath inlet channels. The channel can be part of a particle or cell focusing chip, and the two air channels can be part of a pneumatic chip, where the particle or cell focusing chip is disposed within the pneumatic chip. The single particle or cell dispenser can further comprise a sheath inlet disposed in the particle or cell focusing chip.

The system can further comprise a computing node including a processor and a memory, the memory including instructions that, when executed by the processor, cause the processor to obtain the image of the holographic pattern and/or the fluorescent pattern of the particle or cell; process the image to determine a particle or cell type based on the holographic pattern and/or the fluorescent pattern of the particle or cell depicted in the image; and responsive to determining that the particle or cell type is a target particle or cell, causing an air compressor connected to the air channel inlet of the two air channels direct the flow of pressurized air to each air channel outlet to cause the breakup and removal of the droplet from the outlet of the channel. The cell can be a circulating tumor cell or an immune cell.

An aspect provides a pressurized air-assisted particle dispensing system comprising a microfluidic device comprising one or more sample inlets for introducing a fluid sample, a waste outlet, a target particle or cell outlet, and a particle or cell imaging area, and a channel or tube connected to the target cell or particle outlet and to a first end of a dispenser channel; the dispenser channel comprising a first end and a second end comprising an outlet configured to form a droplet comprising a target cell or target particle and output the droplet; two air channels disposed along a portion of the dispenser channel adjacent to the dispenser channel, wherein the two air channels are configured to obtain a flow of pressurized air at an air channel inlet common to the two air channels and output the flow of pressurized air to an air channel outlet for each of the two air channels, wherein the output of the flow of pressurized air at each air channel outlet is configured to cause a breakup and removal of the droplet from the outlet of the dispenser channel. The system can further comprise an imaging system comprising at least one light source configured to emit a light onto the cell or particle imaging area to highlight a holographic and/or fluorescent pattern of a cell or particle in the cell or particle imaging area; and at least one image sensor configured to capture an image of the particle or cell. The system can further comprise a mechanism to guide the particle or cell to the waste outlet or to the target particle or target cell outlet based on the image of the particle or cell. The system can further comprise a controllable switch configured to engage a compressor to send pressurized air through the two air channels when a target cell or particle reaches the second end of the dispenser channel. The controllable switch can further be configured to engage a vacuum to extract excess liquid where a droplet at the second end of the dispenser channel does not contain a target particle or cell. The image can be an image of the holographic and/or fluorescent pattern of the cells. The system can further comprise a waste tube connected to the waste outlet. The waste tube connected to the waste outlet can be controlled by a valve. The channel or tube connected to the target particle or cell outlet and to a first end of a dispenser channel can be controlled by a valve. The system can further comprise an inlet channel or tube connected to the one or more sample inlets. The inlet channel or tube can be controlled by a valve.

An aspect provides a method of dispensing a droplet with a particle or cell comprising applying a fluid sample to the one or more sample inlets of the pressurized air-assisted particle dispensing system described herein; emitting, by a laser light source, a laser light onto the cell or particle imaging area to highlight a holographic pattern and/or a fluorescent pattern of the particle or cell in the cell or particle imaging area; capturing, by an image sensor, an image of the holographic pattern and/or the fluorescent pattern of the particle or cell; processing, by a computing node, the image to determine a particle or cell type based on the holographic and/or fluorescent pattern of the particle or cell depicted in the image; and responsive to determining that the particle or cell type is a target particle or cell type, causing an air compressor connected to the air channel inlet common to the two air channels to direct a flow of pressurized air to the air channel outlet for each of the two channels to cause breakup and removal of a droplet including the target particle or cell from the outlet of the dispenser channel. The method can further comprise, when no cell or particle is present in the cell or particle imaging area, causing a vacuum pump connected to the air channel inlet common to the two air channels to vacuum excess liquid back into the two air channels and away from the outlet of the dispenser channel. The method can further comprise, responsive to determining that the particle or cell type is a non-target particle or cell type, causing the particle or cell to the waste outlet.

An aspect provides a single cell dispensing system comprising a microfluidic device comprising one or more sample inlets for introducing a fluid sample, a waste outlet, a dispenser channel having a target particle or cell outlet, a particle or cell imaging area, and a pipette air inlet; the target particle or cell outlet is configured to form a droplet comprising a target cell or target particle and output the droplet; two air channels disposed along a portion adjacent to the dispenser channel, wherein the two air channels are configured to obtain a flow of pressurized air at an air channel inlet common to the two air channels and output the flow of pressurized air to an air channel outlet for each of the two air channels, wherein the output of the flow of pressurized air at each air channel outlet is configured to cause a breakup and removal of the droplet from the outlet of the dispenser channel. The pipette air inlet can be configured to be pressurized to prevent backflow and upon detection of a target particle or cell in the imaging area can be pressurized to push a target particle or cell to the target particle or cell outlet. The sample inlet and waste outlet can be connected to a vacuum source. The single cell dispensing system can further comprise an imaging or sensing system comprising at least one light source configured to emit a light onto the cell or particle imaging area to highlight a holographic and/or fluorescent pattern of a cell or particle in the cell or particle imaging area; and at least one sensor configured to capture an image or signal of the particle or cell. The system can further comprise a controllable switch configured to engage a compressor to send pressurized air through the two air channels when a target cell or particle reaches the particle or cell outlet. The pipette air inlet can be pressurized. The system can further comprise a controllable switch configured to engage a vacuum in the waste inlet and sample inlet until a target cell or particle is detected in the particle or cell imaging area, such that the vacuum is turned off and the pipette inlet becomes pressurized thereby moving the target particle or cell to the particle or cell outlet.

An aspect provides a method of dispensing a droplet with a particle or cell comprising applying a fluid sample to the one or more sample inlets of the single cell dispensing system as described herein, emitting, by a laser light source, a laser light onto the cell or particle imaging area to highlight a holographic pattern and/or a fluorescent pattern of the particle or cell in the cell or particle imaging area; capturing, by an image sensor, an image of the holographic pattern and/or the fluorescent pattern of the particle or cell; processing, by a computing node, the image to determine a particle or cell type based on the holographic and/or fluorescent pattern of the particle or cell depicted in the image; and responsive to determining that the particle or cell type is a target particle or cell type turning off the vacuum in the waste inlet and sample inlet. The processing can further comprise, responsive to determining that the particle or cell type is a target particle or cell type, causing an air compressor connected to the air channel inlet common to the two air channels to direct a flow of pressurized air to the air channel outlet for each of the two channels to cause breakup and removal of a droplet including the target particle or cell from the outlet of the dispenser channel.

### BRIEF DESCRIPTION OF THE DRAWINGS

Embodiments of the present invention are illustrated by way of example and not limitation in the figures of the accompanying drawings, in which like references indicate similar elements and in which:

FIG. 1A is a first cross-section view of a pressurized air-assisted particle or cell dispenser of a first example design.

FIG. 1B illustrates an exploded view of a dispenser and an air compressor according to the first example design.

FIG. 1C shows a second cross section view of a dispenser according to the first example design.

FIG. 1D shows a third cross section view of a pressurized air-assisted particle or cell dispenser 100D according to the first example design.

FIG. 2A is a first cross-section view of a dispenser according to a second example design.

FIG. 2B is a rear view of a dispenser 200B according to the second example design.

FIG. 2C is a second cross-section view 200C of a dispenser according to the second example design.

FIG. 3 illustrate various views of a dispenser according to a third example design.

FIG. 4A is a first view of an example system including a dispenser and an imaging setup according to an embodiment.

FIG. 4B is a second view of an example system including a dispenser and an imaging setup according to an embodiment.

FIG. 5 illustrates an example 500 depiction of a droplet and a targeted cell in a glass needle.

FIG. 6 is a flow process of an example method for dispensing a cell using a pressurized air-assisted particle or cell dispenser according to an embodiment.

FIG. 7 is a block diagram of a special-purpose computer system according to an embodiment.

FIG. 8 shows an imaging system and particle or cell dispensing head of a pressurized air-assisted cell dispensing device.

FIG. 9A-9B. FIG. 9A shows a front view of a particle or cell dispensing head showing pinch valves, a microfluidic chip where the target is identified and the pressurized air-assisted particle or cell dispenser for dispensing particle or cells into a designated well or container. FIG. 9B shows the back view of the particle or cell dispensing head.

FIG. 10 shows a microfluidic chip for detection of target cells or particles and delivery of target cells or particles to the dispenser.

FIG. 11A-B shows a bidirectional pressurized air-assisted particle or cell dispenser. FIG. 11A shows a dispenser in push mode where a droplet is dispensed. FIG. 11B shows a dispenser in suction mode where any extra liquid is removed.

FIG. 12 shows an example of a particle or cell dispensing head.

FIG. 13 shows an example of a pressurized air-assisted particle or cell dispenser.

FIG. 14 shows a single cell isolating device with a pressurized pipette line.

### DETAILED DESCRIPTION

Single cell isolation can include separating cells in a biological fluid and isolating individual cells for further processing. Single cell isolation can be used a prerequisite for clonal cell line development and for effective clonal cultivation and omic profiling at single cell level.

The present embodiments generally relate to a microfluidic approach to achieve high precision single particle or cell dispensing for various omic analyses (or other applications such as cell isolation, cell line development, monoclonal antibody production, single-cell multi-omics, characterizing rare cell types). Particularly, in comparison to other single cell dispensing systems, the present embodiments introduce compressed air to reduce the dispensing droplet size and control the uniformity and timing of the droplet generation and dispensing. Furthermore, imaging methods can be used to determine the droplets with targeted particle or cells and assist the dispensing of the droplets into the selected particle or cell collecting containers.

The present embodiments provide different designs of a pressurized air-assisted particle or cell dispenser (also referred to as a particle or cell dropper hereafter) described in detail below. A pressurized air-assisted particle or cell dispenser can include a liquid channel for transporting liquid that carries particle or cell samples to form droplets and compressed air lumens that blow the air from above the droplets formed at the exit of liquid channel to trigger the breakup and pinch-off of the droplets. A single or dual imaging system involving brightfield, phase contrast, fluorescent, and/or holographic imaging can be used to monitor the particles or cells entering the droplets. See, e.g., U.S. Pat. No. 63/482,913, filed on Feb. 2, 2023, and PCT/US24/14321, filed Feb. 2, 2024 which are incorporated herein in their entirety. The imaging signals can be used to control the position of the particle or cell collecting containers to ensure each individual targeted particle or cell is correctly dispensed.

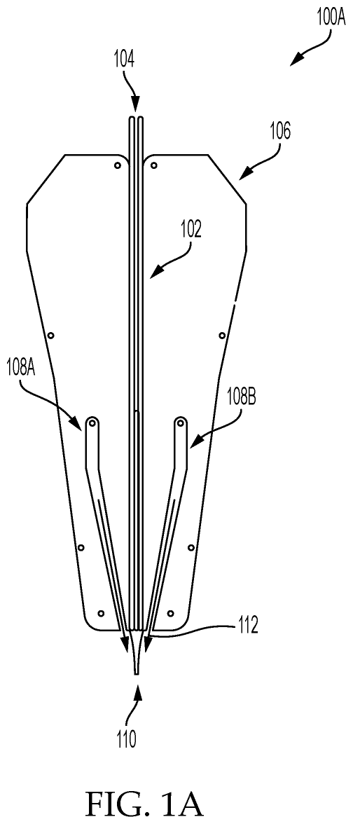

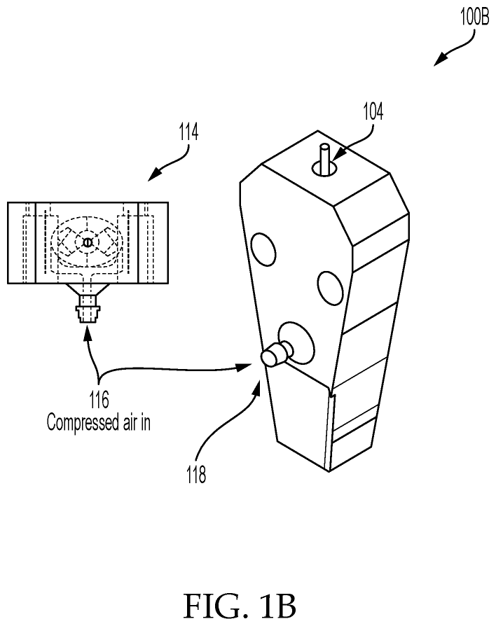

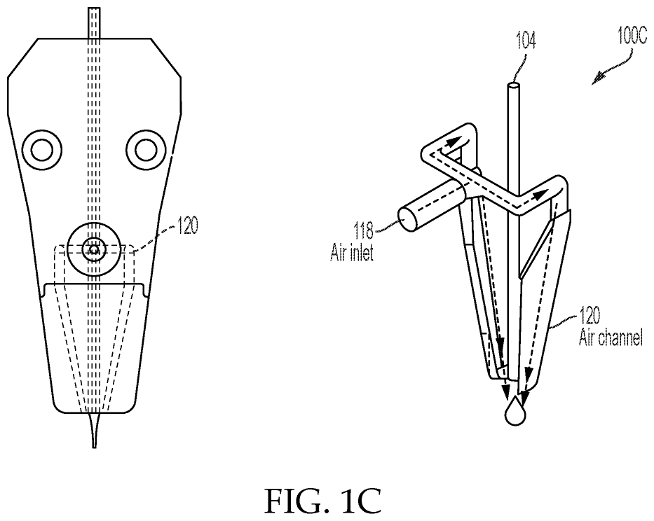

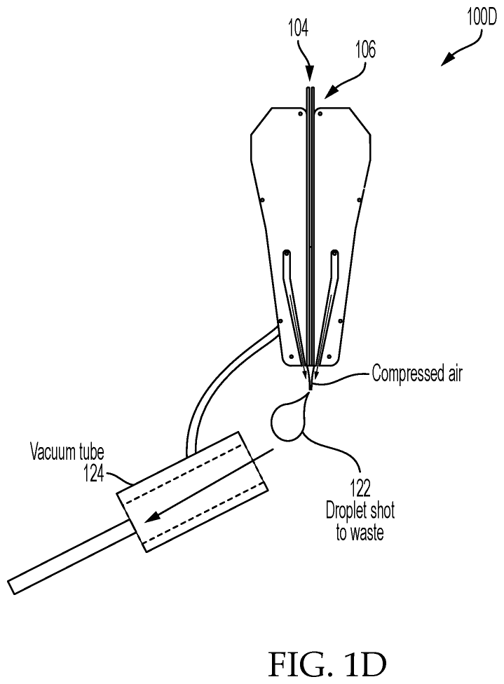

FIGS. 1A-1C illustrate a pressurized air-assisted particle or cell dispenser according to a first example design. For instance, the dispenser as shown in FIGS. 1A-1C can illustrate a dispenser design with a capillary tube for liquid sample transport and two symmetrically located pressurized air channels for droplet size control. Furthermore, the figures can illustrate the air lumens inside the device with a liquid flow and air channels for which compressed air aligns itself with the droplet

FIG. 1A is a first cross-section view of the pressurized air-assisted particle or cell dispenser of the first example design. As shown in FIG. 1A, the dispenser 100A can include a tubular channel 102 (or a main channel) disposed through a main body 106 of the dispenser 100A. One or more particle or cells can be input at a particle or cell inlet 104 to be directed along the tubular channel 102 to an outlet 110.

Furthermore, the dispenser can include both a liquid flow from the tubular channel and multiple air flow channels. For example, as the particle or cell travels along the tubular channel 102 to the outlet 110, multiple air channels 108A-B can direct compressed air 112 at a point near the outlet 110.

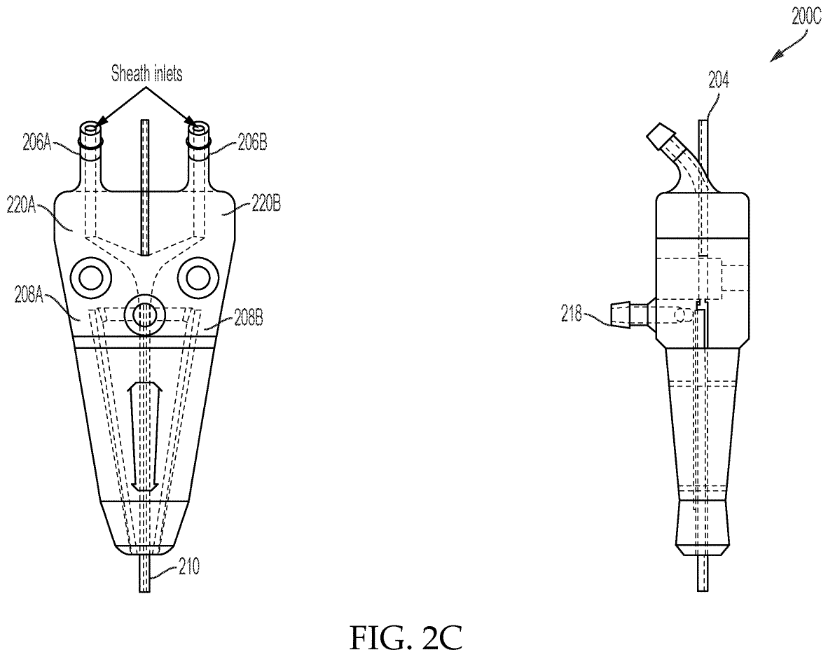

Liquid carrying particle or cell samples can flow in a channel 102 such as a tubular channel (e.g., a glass capillary tube or other suitable tube or channel) with an inner diameter between 10 μm to 300 μm (e.g., about 10, 10, 100, 150, 200, 250, 300 μm). The tubular channel can be a straight tube with constant diameter, or a tube that tapers to a smaller diameter at the end. Furthermore, two compressed air lumens (e.g., air channels 108A-B) can be located symmetrically on two sides of the tubular channel and oriented in a shallow angle with respect to the tubular channel. The air channels can run parallel to the tubular channel and then angle towards the dispenser outlet. See, e.g., FIG. 3.

For all embodiments described herein, the air channels can be any cross-sectional shape, e.g. circular, oval, square, rectangular, etc. The air channels can be about 200 μm to 2 mm (e.g., about 200, 300, 400, 500, 600, 700, 800, 900, 1,000, 1,500, 2,000 μm or more) in diameter or can have side walls of about 200 μm to 2 mm. (e.g., about 200, 300, 400, 500, 600, 700, 800, 900, 1,000, 1,500, 2,000 μm or more). Alternatively, the air channels can run at an angle towards the dispenser outlet. In either aspect the angle can be about 5, 10, 15, 20, 30, 35, 40, 45 degrees or more offset from the tubular channel. The compressed air can trigger the breakup and pinch-off of the droplet from the exit of the tubular channel. For all embodiments described herein, the outlet of the air channels can be within about 0.1, 0.5, 1.0, 2.0, 3.0, 4.0, 5.0, 6.0, 7.0, 8.0, 9.0, 10.0 mm or more from the dispenser outlet. Where two air outlets are present, they can each be the same distance from the dispenser outlet, or can be different distances. For all embodiments described herein, the combination of liquid flow rate and the compressed air flow rate can determine the droplet size and droplet generation rate. For all embodiments described herein, a liquid flow rate can be about 0.1 μL/min up to 1 mL/min (e.g., about 0.1, 0.5, 1.0, 5.0, 10, 50, 100, 250, 500, 750, 1,000 μL/min or more. For all embodiments described herein, a compressed air flow rate can be about 1 mL/S to 100 mL/S (e.g., about 1, 2, 5, 10, 25, 50, 75, 100 or more mL/S. For all embodiments described herein, the droplet generation rate can be about 1, 2, 3, 4, or more drops per second or about 1, 2, 3, 4, 5, 6, 7, 8, 9, or 10 drops per 1, 2, 3, 4, 5, 6, 7, 8, 9, 10 more seconds. For all embodiments described herein, droplet size can be about 10 μm to about 800 μm (e.g., 50, 100, 150, 200, 250, 300, 350, 400, 450, 500, 550, 600, 650, 700, 750, 800 μm or more.

For example, the pressurized air-assisted particle or cell dispenser can include a tubular channel (e.g., 102 in FIG. 1A). The tubular channel can include a first end and a second end, the first end can include an inlet (e.g., 104) to obtain a liquid containing a particle or cell. The second end can include an outlet (e.g., 110) configured to form a droplet comprising the particle or cell and output the droplet.

The dispenser can also include at least one air channel (e.g., 108A-B) disposed along a portion of the pressurized air-assisted particle or cell dispenser adjacent to the tubular channel. The at least one air channel can include an air channel inlet (e.g., 118) configured to obtain a flow of pressurized air and at least one air channel outlet adjacent to the outlet of the tubular channel. The output of the flow of pressurized air at the air channel outlet can be configured to cause a breakup and removal of the droplet from the outlet of the tubular channel.

FIG. 1B illustrates an exploded view 100B of the dispenser and an air compressor 114. The air compressor 114 can provide pressurized (or compressed) air 116 to the air inlet 118. The air compressor 114 can be directly connected to the dispenser or connected to the dispenser via one or more tubes. The voltage of the air compressor can control the flow rate. A flow controller can be used to control the flow rate.

FIG. 1C shows a second cross section view of the dispenser 100C. As shown in FIG. 1C, air channels 120 can be disposed in the dispenser. For example, air can be directed in an air inlet 118 and directed along two air channels (e.g., 120) to be output as described herein.

A second dispenser design can include a three-inlet channel with symmetric sheath flow for liquid sample transport and two symmetrically located pressurized air channels for droplet size control. The droplet size can be controlled by the air flow rate. Generally, a higher flow rate can reduce a critical size for the droplet to break at the outlet. Further, the air pressure can add to the weight of the droplet and overcome the surface tension to remove from the outlet.

FIG. 1D shows a third cross section view of a pressurized air-assisted particle or cell dispenser according to the first example design. As shown in FIG. 1D, the compressed air at the outlet can break the droplet from the device. Further, the droplet can be shot (e.g., 122) to a waste collector via a vacuum tube 124. The vacuum tube 124 can be used to remove unwanted droplets (e.g., droplets including particle or cells not of the target particle or target cell type). The header of the device can include a small tube close to the tubular channel outlet tip. Whenever the droplet is considered as waste, the air vacuum pump can act (e.g., turn off and on or open and close a valve) to remove the unwanted droplet from the needle's tip. The same vacuum droplet extraction design can be applied to any of the other designs as described herein for removing unwanted droplets.

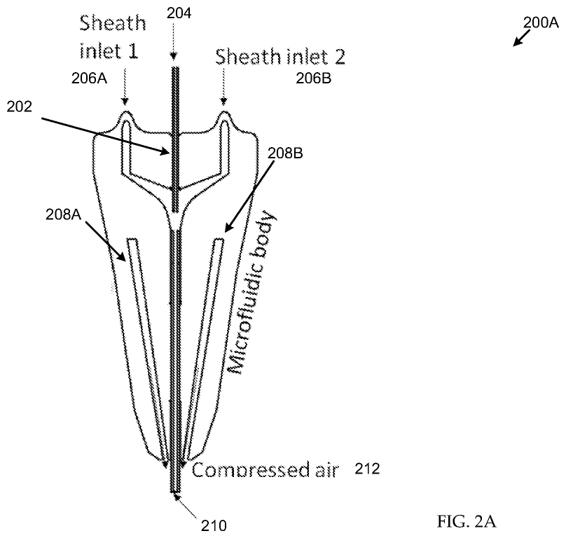

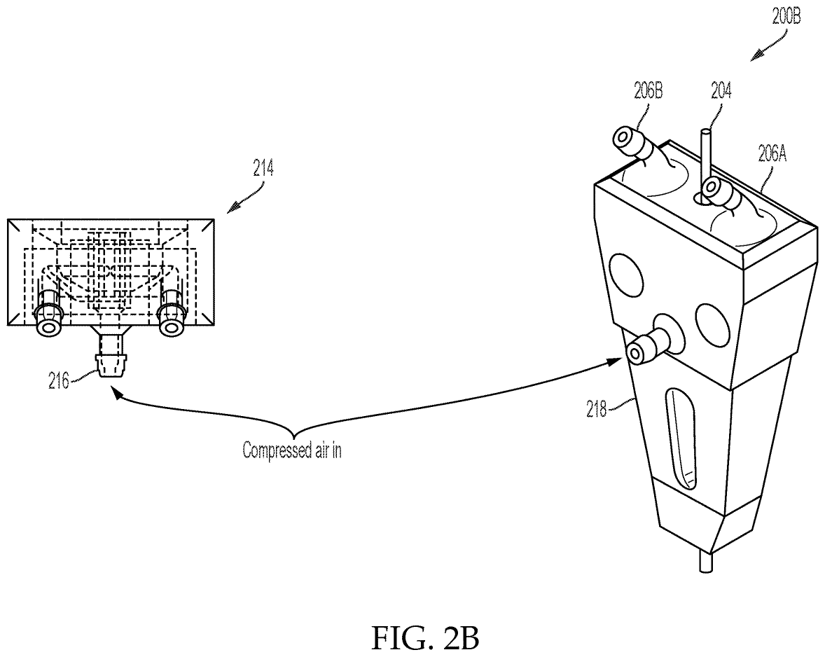

FIGS. 2A-2C illustrate a pressurized air-assisted particle or cell dispenser according to a first example design. FIGS. 2A-2C can illustrate the design with a three-inlet channel with symmetric sheath flow for liquid sample transport and two symmetrically located pressurized air channels for droplet size control.

FIG. 2A is a first cross-section view of a dispenser according to a second example design. As shown in FIG. 2A, the dispenser 200A can include tubular channel 202 with an inlet 204 and an outlet 210. Furthermore, the dispenser 200A can include multiple (e.g., 2, 3, 4, or more) sheath inlets 206A-B. The sheaths 206A-B can direct liquid flow along channels formed by the sheaths to focus the position of the particle or cell as the particle or cell travels down the tubular channel 202. The liquid can be sample liquid or another suitable liquid like saline. The sheath liquid flows around a secondary flow to keep it focused. 206A and 206B are sheath inlets of the same chamber that can narrows as they goes downward to create a sheath flow around the flow that is coming from 204. Now the sample from 204 has the sheath from 206A, B and flows into the 210. 208A and 208B are connected to the pressured air source. This guides the air flow to blow toward the tip of the dispenser and push the droplet out of the dispenser. The dispenser 200A can also include multiple air channels 208A-B for directing pressurized air 212 at the outlet 210.

In the second example design, the liquid carrying particle or cell samples can flow in a three-inlet channel with a sheath flow (e.g., at inlets 206A-B) to focus the stream of particle or cells to desired stream widths (e.g., 10, 50, 100, 150, 200, 250, 300 μm or more). The particle or cell focusing can be controlled by adjusting the flow rates of sample stream and sheath flow, with any of the sheath flow and/or stream ranging from 1-50 μL/min (e.g. about 1, 5, 10, 15, 20, 30, 40, 50 μL/min). Two pressurized air lumens can be located symmetrically on two sides of the tubular channel and oriented in a shallow angle with respect to the liquid flow channel. In comparison with the first example design, the sheath flows can allow for use of larger diameter tubular channels for transporting liquid sample while keeping the particle or cells focused in the center of the tube for imaging purposes. The second example design can reduce the chances of particles, cells, or cell clusters clogging the flow channel, which can be a common problem when smaller diameter tubular channels are used.

FIG. 2B is a rear view of the dispenser according to the second example design. As shown in FIG. 2B, the dispenser can include a tubular channel inlet 204 and sheath inlets 206A-B. Furthermore, an air compressor 214 can provide pressurized air 216 to an inlet 218.

FIG. 2C is a second cross-section view of the dispenser according to the second example design. As shown in FIG. 2C, the sheath inlets 206A-B can direct fluid (e.g., sample fluid or other suitable fluid e.g., saline) along sheath paths 220A-B. Furthermore, air channels 208A-B can direct pressurized air (e.g., compressed by compressor 214) input at input 218.

In some instances, the sheath can include a fluid containing oil. Oil droplets can encapsulate one or more particle or cells. Furthermore, the sheath can include molecular barcodes configured to tag particle or cells in a droplet. See, e.g., Kim, Single-Cell Molecular Barcoding to Decode Multimodal Information Defining Cell States. Mol Cells. 2023 Feb. 28; 46(2): 74-85. For example, oil can be added from a channel (e.g., a sheath flow channel) which comes in contact with sample cells. The cells become trapped in oil at the junction of the inlet channel and the sheath channel. Oil droplets then flow suspended the sample liquid. The oil contains a barcode or tag particle, which can be a microparticle that is used for different purposes. For example, it can extract the cell's RNA for example which is useful for RNA sequencing.

A third example dispenser design provides a microfluidic chip for focusing particle or cell sample stream and a pneumatic chip for injecting pressurized air to control droplet size. FIG. 3 illustrate various views of a dispenser 300 according to a third example embodiment. As shown in FIG. 3, the dispenser can include air inlets 302A-B and a particle or cell inlet 308. The dispenser can include a microfluidic chip comprising a pneumatic chip 304 and a particle or cell focusing chip disposed within the pneumatic chip 304. The particle or cells input at inlet 308 can be directed down a channel to an outlet (e.g., droplet generation 310). Furthermore, the air directed in from the air inlets 302A-B to the outlet can cause an air flow 314 at the outlet (e.g., 310) to push the droplet.

In the third example dispenser design, the liquid carrying particle or cell samples can flow in a transparent microfabricated particle or cell focusing chip 312 (e.g., a depth between 10 μm to 300 μm, e.g., about 10, 50, 100, 150, 200, 250, 300 μm or more) with sheath flow from a sheath inlet 306 to focus the stream. The particle or cell focusing can be controlled by adjusting the flow rates of sample stream and sheath flow. The particle or cell focusing chip can be placed inside a pneumatic chip with two air inlets. The air coming out of the pneumatic chip can impinge symmetrically from the two sides of a droplet and can trigger its breakup. The channels for transporting particle or cell samples and for compressed air injection can be separated. In some aspects, the inner chip for transporting particle or cell samples can be frequently replaced to ensure the sterility of the sample while the outer pneumatic chip does not require frequent replacement.

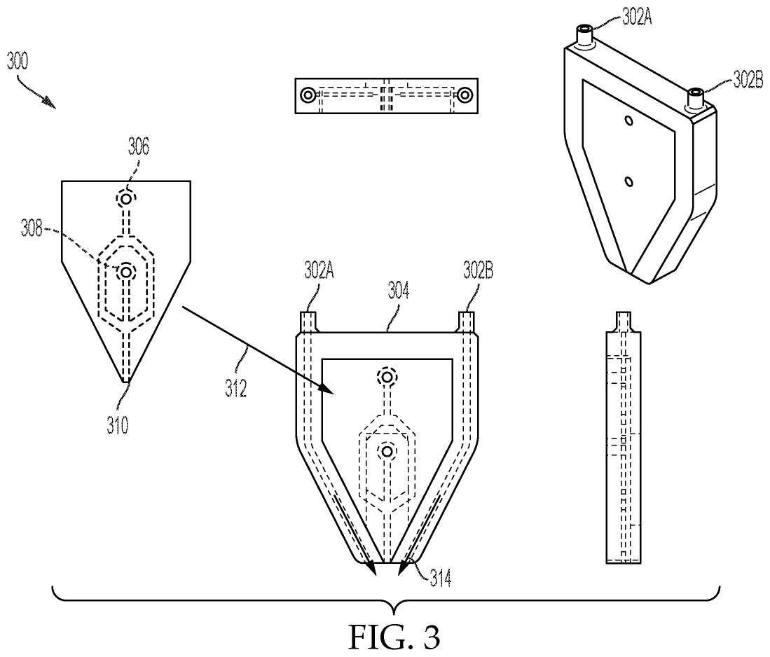

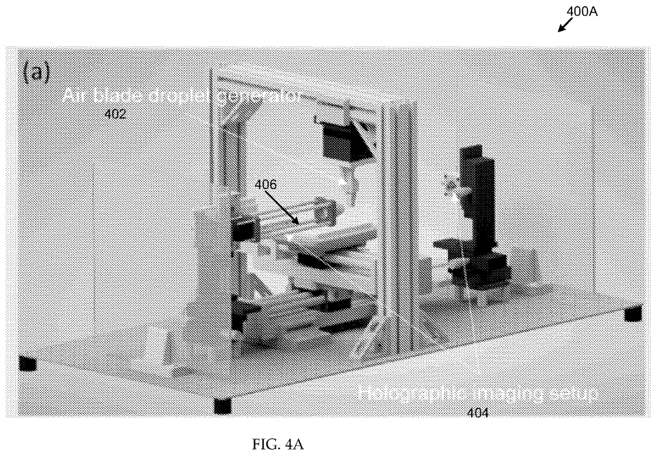

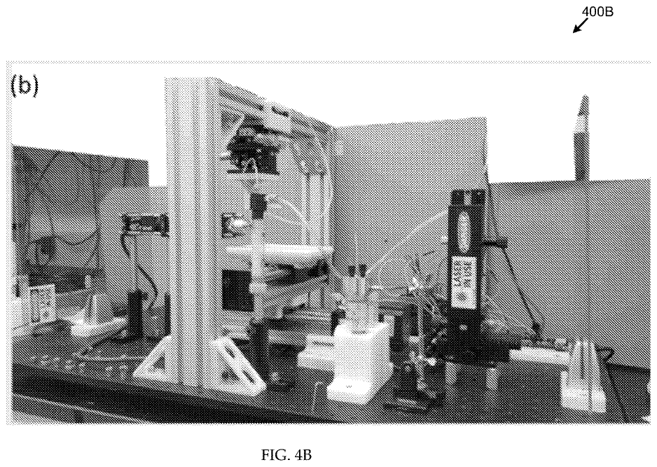

FIGS. 4A-4B illustrate an example system including a dispenser and an imaging setup. For example, as shown in FIG. 4A, the system 400A can include a pressurized-air-assisted droplet generator 402, which can include features of any dispenser as described herein. The system 400A can further include a holographic imaging setup 404 (or other suitable imaging set up, e.g., fluorescent, bright field, phase contrast), which can include one or more image sensors (or cameras) configured to capture one or more images (e.g., 1, 2, 3, 4, or more images) of a droplet. The droplet provided by the pressurized air-assisted droplet generator 402 can be dropped into a well in a tray 406. In some instances, the imaging setup 404 can capture images of the droplet, and a computing node (or series of interconnected computing nodes) can determine whether the particle or cell in the droplet is a particle or cell of interest. Based on determining that the particle or cell is a particle or cell of interest, the particle or cell can be deposited in a first well in the tray 406. If the particle or cell is not of interest, the particle or cell can be deposited into a waste collection container. FIG. 4B illustrates an implementation of the system (e.g., 400B) as shown in FIG. 4A.

In some instances, the system as described in FIGS. 4A-4B can use a dispenser (e.g., the dispenser as described in design 1) for droplet generation and particle or cell dispensing. The particle or cell dropper can be fabricated using, e.g., 3D printing. A pressure pump can be used to transfer a fluid that contains a sample, such as a cell sample containing, for example, circulating tumor cells (CTCs), blood cells, immune cells, cells labeled with a tag (e.g., a fluorescent tag), barcoded cells etc., into the inlet of the cell dropper. When the pump operates at a low flow rate, droplets can be generated at the outlet of a tubular channel (e.g., a glass needle) and fall vertically due to gravity. The rate of droplet generation and the size of droplets can depend on the flow rate, the needle tip diameter, fluid properties (e.g., surface tension coefficient and viscosity), and/or surface properties of the needle. The particle or cell dropper can include 1, 2, 3, 4, or more embedded air channels which are connected to the air pump that can supply pressurized air going through the channel. The pressurized air can be filtered.

The activation of the pressurized air through the air channels can cause the early breakup of the droplets at the needle tip and can leads to the formation of smaller droplets. During the operation of the system, the tubular channel or dispenser channel can be coated with hydrophobic coating to assist the formation of small droplets. The tubular channel or dispenser tip (e.g., a glass needle tip) diameter (from 10 μm to 300 μm, e.g., about 10, 50, 100, 150, 200, 250, 300 μm or more) and the flow rate of the pressurized air can be adjusted to control the droplet size ranging from 50 μm (corresponding to about 60 pL) to 800 μm (corresponding to about 250 nL) (e.g., about 50, 100, 200, 300, 400, 500, 600, 700, 800 μm or more) and droplet generation rate ranging from one droplet per two seconds to one droplet per 10 seconds (e.g., about 1, 2, 3, 4, 5, 6, 7, 8, 9, 10 or more droplets per 1, 2, 3, 4, 5, 6, 7, 8, 9, 10 or more seconds or any combination thereof). This adjustment can accommodate the dispensing of particle, cells, and cell clusters (e.g., CTCs and CTCs clusters) across different size ranges and at different levels of purity. For example, to dispense single cell clusters of 50 to 80 μm, the needle tip can be 100 μm in diameter and the air flow rate (e.g., 0.5 mL/s to 100 mL/s) to generate 400 μm diameter droplet at a rate of one droplet per five seconds.

A holographic imaging system (e.g., 404) can use a digital inline holographic (DIH) setup. See e.g., WO2021/155322 and U.S. Pat. Publ. 20230040252, which are incorporated herein by reference in their entirety. The DIH setup can include, e.g., a 405 nm 10 mW diode laser as the light source and a microscopic imaging module comprising a 10x microscopic objective and FLIR Blackfly S 5M monochromatic camera. The laser and the imaging module can be mounted on a 3D translation stage and can be located on either side of the particle or cell dropper. The laser can pass through the tubular channel (e.g., a glass needle) and illuminate the particle or cells inside to generate particle or cell holograms captured by the imaging module. The imaging sample volume of about 600 μm×500 μm×400 μm can be centered about 30, 40, 50, or 60 μm upstream of the tubular channel outlet (e.g., a needle tip), which allows monitoring the particle or cells going into the droplet and the droplet generation process. The obtained holograms, though distorted slightly due to the curvature of the tubular channel (e.g., a glass needle), can allow for determining whether a particle or cell is transported into a droplet and when the droplet is detached from the outlet of the tubular channel. The motorized translation stage can include two linear stepper motors with, for example, 30 μm precision (i.e., the minimal step of its movement) used to control the x and y translation, respectively and a mounting stage on top for mounting a standard multi-well plate.

With respect to digital holographic microscopy, digital holography can be used to record a wave front diffracted from an object by a light source. Utilizing the interference of light from the light source, both amplitude and phase information of an object wave can be recorded to produce a hologram containing the information of the object wave. A three-dimensional image can then be reconstructed from the hologram.

In an embodiment utilizing digital holographic microscopy, a light source can comprise various types of illuminating devices, such as but not limited to a laser such as a monochromatic laser. A pair of laser light waves can be generated from the light source by dividing the laser beam with a beam splitter such that one of the split light waves illuminates the biological fluid. The light diffracted from the biological fluid can form an object wave, which illuminates the light source and is collected by the microscope objective. The remaining laser light wave can be directly detected by the microscope objective to serve as a reference wave. The object and reference waves can interfere with each other to form an interference fringe image which is scanned by an image sensor. In some embodiments, a single laser beam can be used. A portion of the laser beam can be scattered by the biological sample and unscattered portion of the laser can serve as the reference light. Holograms can be generated from the interference between the scattered light and reference light.

Continuing to reference digital holographic microscopy, the object and reference wave fronts can be joined by the beam splitter such that the object and reference wave fronts interfere and create a hologram which can be detected by an image sensor. The computing node can then process the digital hologram, with the computing node functioning as a digital lens to calculate a viewable image of the object wave front utilizing a numerical reconstruction algorithm.

Digital holographic microscopy can be utilized to observe particles or living cells within the biological fluid. From the recorded interference pattern of such living cells, the intensity and phase shift across various points of the cells can be numerically computed by the computing node. The computing node can thus measure the phase delay images of biological cells within the biological fluid to provide quantitative information about the morphological properties (e.g., cellular dry mass, surface texture, shape, etc.) of individual cells within the biological fluid. By way of example and without limitation, the computing node can be adapted to extract parameters such as cell thickness, cell area, cell volume, cell dry mass, the phase shift across the cell, surface roughness and texture, cell shape, elongation, convexity, luminance, circularity, solidity, and the like.

Various types of digital holography can be utilized with the systems and methods described herein, including but not limited to off-axis Fresnel, Fourier, image plane, in-line, Gabor, and phase-shifting digital holography. By utilizing digital holographic microscopy, the computing node can differentiate between the various constituents within a biological fluid sample for further processing utilizing the systems and methods described herein. It should be appreciated that multiple laser wavelengths can be utilized when scanning the biological fluid with digital holographic microscopy. It has been shown that the refraction amount increases as the wavelength of light decreases. Thus, shorter wavelengths of light (e.g., violet and blue) are more slowed and consequently experience more bending than longer wavelengths of light (e.g., orange and red).

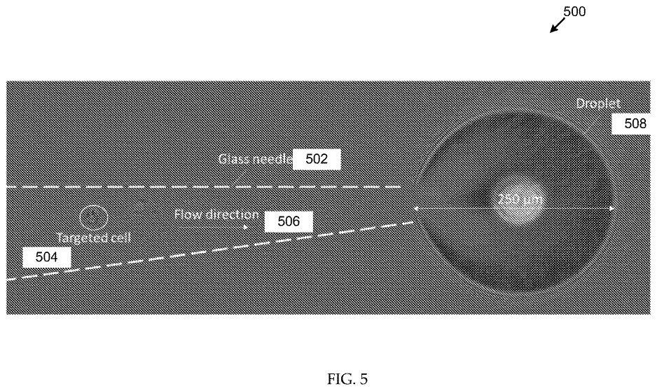

FIG. 5 illustrates an example depiction of a droplet and a targeted particle or cell in a tubular channel that can be, e.g., a glass needle. As shown in FIG. 5, the glass needle 502 can direct particle or cells (e.g., targeted particle or cell 504) in a flow direction 506 toward the droplet 508. FIG. 5 illustrates a sample hologram showing the droplet generation at the exit of the tubular channel and the transport of targeted particle or cell into the droplet. The targeted particle or cell can be disposed in the droplet, and the imaging system can determine that the targeted particle or cell is included in the droplet and can instruct the dispenser to drop the droplet into a well for further analysis.

In an aspect, fluorescent, bright field, or phase contrast imaging can be used to detect cells and particles. In bright field imaging, sample illumination is transmitted with white light (therefore, the light source can be white light) and contrast in the sample is caused by attenuation of the transmitted light in dense areas of the sample. In phase contrast microscopy, partially coherent illumination produced by a lamp, e.g., a tungsten-halogen lamp is directed through a collector lens and focused on a specialized or condenser annulus positioned at a substage condenser front focal plane. Wavefronts passing through an annulus illuminate the sample and either pass through undeviated or are diffracted and retarded in phase by structures and phase gradients present in the sample. Undeviated and diffracted light collected by the objective is segregated at a rear focal plane by a phase plate and focused at the intermediate image plane to form the final phase contrast image.

In an aspect, a target particle or cell can be fluorescently labeled. In another aspect, a target particle or cell can be fluorescently labeled and a non-target particle or cell can be differentially fluorescently labeled. A light source for imaging can be, for example, white light, laser light, LED, xenon arc lamps, tungsten lamps, halogen lamps, fluorescent light, UV light, etc. The wavelengths of the light provided can range between, for example, 350 nanometers (nm) and 800 nm (e.g., 350, 400, 450, 500, 550, 600, 650, 700, 750, or 800 nm). Cameras used for capturing imaging modalities beyond DIH (e.g., fluorescence) can be replaced with other types of light sensors. This can include sensors such as photomultiplier tubes, photodiodes, and other sensors. Such sensors can be used to improve the sensitivity and/or sampling rate of the measurements.

The one or more cameras that are part of the imaging system can capture one or more images (or video) of the particle or cell in the tubular channel. A laser light source can emit a light into the tubular channel to highlight a holographic, fluorescent, bright field, or phase contrast pattern indicative of the particle or cell type. The image(s) can be provided to a computing node for processing to determine a particle or cell type of the targeted particle or cell. For example, the holographic, fluorescent, bright field, or phase contrast pattern can be processed to determine a particle or cell type (e.g., a circulating tumor cell (CTC), a white blood cell, red blood cell, etc.). If the particle or cell type is a type of interest (e.g., a CTC cell), the computing node can cause the flow of pressurized air to flow along the air channels to break up and output the droplet into a collection container (e.g., a well part of a multi-well tray such as a microtiter plate). If the particle or cell is not of interest, the particle or cell can be output into a waste container. For example, if the particle or cell is not of interest, the droplet that contains the particle or cell can be disposed to a waste collector. The shift between the waste collector and sample collector can be achieved by an x-y translation stage. When the target particle or cell is detected, the sample collecting well can be translated right below the particle or cell dispenser, which can allow the droplet to fall into the collector. Alternatively, the cell dispenser can be present on a x-y translation stage and can be moved to dispense the target particle or cell to the sample collector or to the waste collector.

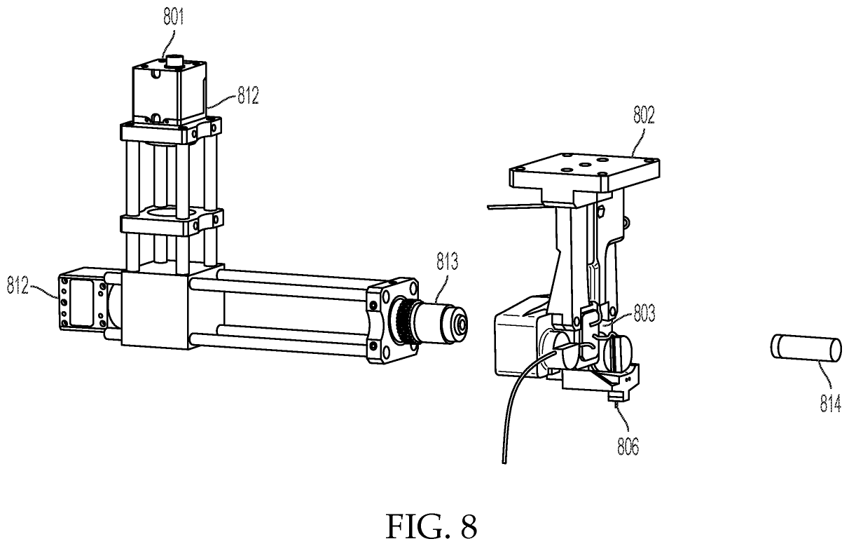

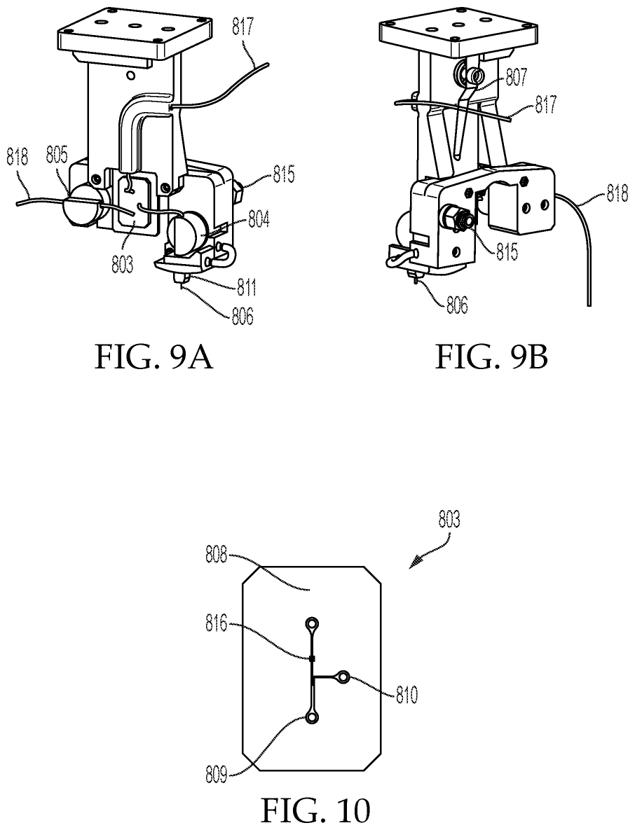

Bidirectional Pressurized Air-Assisted Particle or Cell Dispenser Another aspect provides a bidirectional pressurized air-assisted particle or cell dispenser. A bidirectional pressurized air-assisted particle or cell dispenser can set the size of droplets. A dispenser can be engineered for efficient and accurate isolation of specific cells or particles in a microfluidic environment. In an aspect a droplet of a desired volume containing one target particle or cell can be dispensed. A particle or cell dispenser system can comprise an imaging module 801. See FIG. 8. An imaging module can include one or more image sensors (e.g., cameras, photodiodes, or photomultiplier tubes 812 (PMT)) and combinations thereof) configured to capture one or more images of a droplet to accurately detect and/or identify a target cell or particle within the droplet. The imaging module can comprise one or more objectives 813. A particle or cell dispenser system can comprise a particle or cell dispensing head 802. See FIG. 9. A particle or cell dispensing head can comprise a microfluidic chip holder 807 and a removable/replaceable microfluidic chip 803. The microfluidic chip can be disposable or reusable. One or more light sources, e.g., a laser source 814 can be used to illuminate the microfluidic flow at an imaging area 816 containing target cells or particles.

A microfluidic chip can have one or more (e.g., 1, 2, 3, or more) inlets 808 (e.g., an inlet tube or other suitable inlet) for introducing the sample and one or more outlets (e.g., 1, 2, 3, 4, or more) for directing the flow. See FIG. 10. A sample containing cells or particles enters through an inlet 808, and is then directed to outlets such as a waste outlet 809 or a target cell or particle outlet 810. A particle or cell imaging area on the chip 816 can be present where the cells or particles in the fluid stream are detected by the imaging system. The particle or cell imaging area 816 can occur between the inlet(s) and outlet(s). The target cell or particle outlet can lead to the dispenser 806. One or more valves, such as pinch valves can be present. A dispenser valve 804, such as a pinch valve, and a waste valve 805 such as a waste pinch valve, can be used to control the outlets. Valves can also be present at an inlet tube 817, which feeds the fluid to the microfluidic chip and/or the outlet tube 818, 827 which delivers waste from the waste outlet 809 of the microfluidic chip to a waste container. The inlet and outlet tubes can be external to the microfluidic chip. When the dispenser valve 804 for a target cell or particle is activated, the fluid is sent to the dispenser. Conversely, when the waste valve for the waste is activated, the fluid is directed to the waste outlet tube.

The dispenser channel can have an inner diameter between 10 μm to 300 μm (e.g., about 10, 10, 100, 150, 200, 250, 300 μm). The dispenser channel can be a straight tube with constant diameter, or a tube that tapers to a smaller diameter at the end. Furthermore, two compressed air channels can be located symmetrically on two sides of the dispenser channel and oriented in a shallow angle with respect to the tubular channel. The air channels can run parallel to the tubular channel and then angle towards the dispenser outlet 806. Alternatively, the air channels can run at an angle towards the dispenser outlet 806. In either aspect the angle can be about 5, 10, 15, 20, 30, 35, 40, 45 degrees or more offset from the tubular channel. The compressed air can trigger the breakup and pinch-off of the droplet from the exit of the tubular channel. The combination of liquid flow rate and the compressed air flow rate can determine the droplet size and droplet generation rate. A liquid flow rate can be about 0.1 μL/min up to 1 mL/min (e.g., about 0.1, 0.5, 1.0, 5.0, 10, 50, 100, 250, 500, 750, 1,000 μL/min or more. A compressed air flow rate can be about 1mL/S to 100mL/S (e.g., about 1, 2, 5, 10, 25, 50, 75, 100 or more mL/S. The droplet generation rate can be about 1, 2, 3, 4, or more drops per second or about 1, 2, 3, 4, 5, 6, 7, 8, 9, or 10 drops per 1, 2, 3, 4, 5, 6, 7, 8, 9, 10 more seconds. Droplet size can be about 10 um to about 800 μm (e.g., 50, 100, 150, 200, 250, 300, 350, 400, 450, 500, 550, 600, 650, 700, 750, 800 μm or more.

Dispenser valve activation can be controlled by any suitable method. In certain aspects, a valve triggering unit can be used to generate triggers for the target particle or cell. A valve trigger unit can actuate a device based on the one or more triggers to send the target particle or target cell to a cell or particle outlet 810 of a microfluidic device. The device used to separate the particle or cell of interest from the sample can be a valve or other mechanical device. Charging plates and/or other pneumatic, piezoelectric, and/or electronic devices can also be used to send target particles or cells to a particle or cell outlet 810. See, e.g., U.S. Pat. Publ. 20230040252 entitled Label Free Cell Sorting, which is incorporated herein by reference in its entirety. Separation of target particles or cells (sorted to particle or cell outlet 810) from non-target particles or cells (sorted to a waste outlet 809) can also be accomplished using, e.g., a feedback controlled microfluidic piezoelectric valve. See, WO2023205419A1, entitled Feedback Controlled Microfluidic Piezoelectric Valve, which is incorporated herein by reference in its entirety A particle or cell dispensing head 802 can also comprise a pneumatic microfluidic device 811. This is a specialized device within the system that uses air pressure to precisely control the size of the droplets being formed. It also has the capability to remove any excess liquid, channeling it efficiently to a waste container. A pneumatic microfluidic device 811 can comprise an air inlet 815.

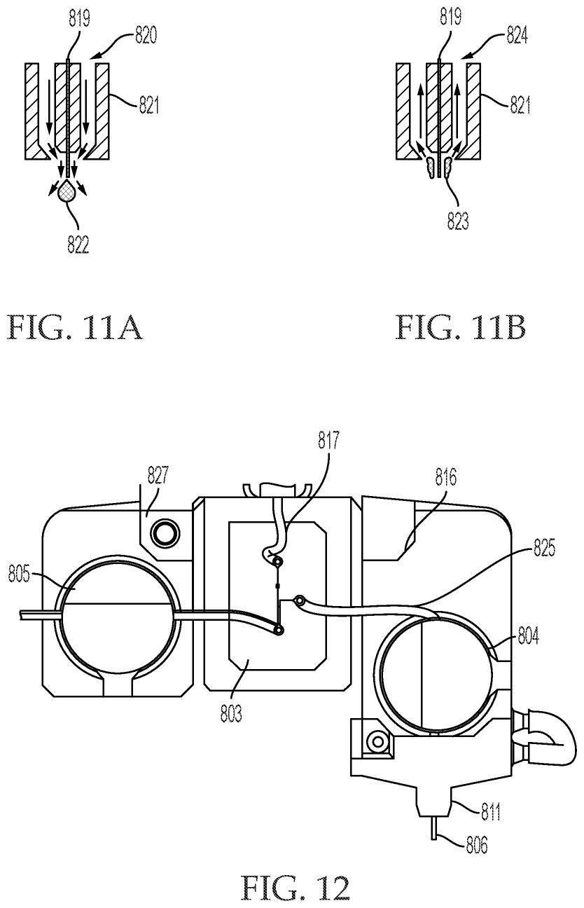

Features of a bidirectional pressurized air-assisted particle or cell dispenser 821 can be integrated into microfluidic channels that are tailored for air flow, which are linked to a controllable switch. See FIG. 11. This switch can control the connection of the microfluidic channel either to a compressor or to a vacuum pump, based on operational requirements. When the target cell or particle is detected in the cell imaging area, and moved to the outlet on the microfluidic chip to a liquid flow to the dispenser 819, the switch engages the compressor to send pressurized air through the air channel to the compressor 820 towards the dispenser, facilitating the propulsion of the droplet 822 containing the target cell or target particle towards the designated target container. Conversely, in instances where the droplet lacks content, the switch activates the vacuum pump, which then efficiently extracts any surplus liquid 823 through the air channel to the vacuum pump 824, ensuring precise volume control within the system.

An additional view of a particle or cell dispensing head is shown in FIG. 12. The sample inlet tube 817 leads to the microfluidic chip sample inlet 808. The fluid sample moves to the imaging area on the chip 816. A non-target cell or particle can be imaged and directed to the waste outlet on the microfluidic chip 809 and through the waste tube 818 controlled by the waste valve 805. A target cell or particle can be imaged and directed to the target particle or cell outlet on the microfluidic chip 810 and through a tube 825 controlled by the dispenser pinch valve 804.

A fluid sample can be pushed into a microfluidic chip by a pump or compressor when a waste valve is closed, and the dispenser valve is open, provided no target particle or cell is detected in the imaging area. This configuration prevents flow towards the dispenser and directs flow towards the waste outlet 809, allowing the sample to flow into the waste tube 818, 827 and to a waste container. Upon detection of a target particle or cell in the imaging area, the waste valve closes, and the dispenser valve opens, guiding the target cell or particle towards the dispenser. The cell or particle speed is determined by capturing two images and calculating the displacement of the cell or particle. See for example, U.S. Pat. Publ. 20230040252, which is incorporated by reference herein in its entirety. Briefly, image data of a sample including multiple particles or cells is collected. The image data can include multiple image frames, wherein each of the multiple image frames includes image data of a portion of the multiple particles or cells included in the sample. A trigger is generated for each of the multiple image frames based on timing data corresponding to a capture time for the image data included in each of the multiple image frames. A frame of interest (e.g., a frame including a target cell or particle) is detected within the multiple image frames based on an output of a classification model. The target particle or cell of interest can be sent to a microfluidic chip outlet by actuating a device based on the trigger for the frame of interest. The device can be, for example, a mechanical, pneumatic, piezoelectric, and/or electronic device.

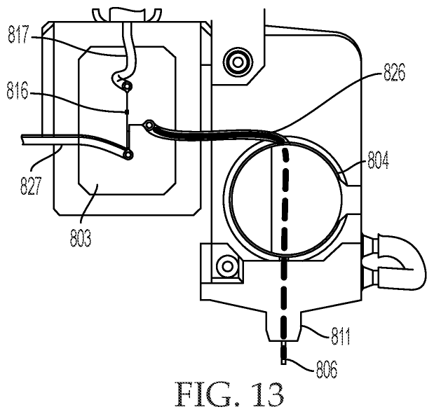

This process helps to estimate the time the target cell or particle will reach the dispenser outlet. The tube connected to the dispenser 825 has an inherent dead volume 826, depending on its inner diameter. See FIG. 13. The dead volume can vary from between 1 μL to 50 μL (e.g., about 1, 5, 10, 20, 30, 40, 50 μL or more). This volume represents the minimum size of the droplet that can be formed at the dispenser's tip, without activation of the bidirectional pressurized air-assisted particle or cell dispenser.

Where a target cell or particle is detected in the particle or cell imaging area a switch (e.g., a pneumatic switch) can connect the air channel to a vacuum source, drawing out the excess volume of the droplet. At the estimated time a target particle or cell is predicted to meet the dispenser, the pneumatic switch can connect the air channel to a compressor, ensuring the target cell or target particle is effectively ejected at the calibrated size (e.g., about 1, 5, 10, 20, 30, 40, 50 μL or more).

Therefore, a bidirectional pressurized air-assisted particle dispensing system can comprise, for example, a microfluidic device comprising one or more sample inlets for introducing a fluid sample, a waste outlet, a target particle or cell outlet, and a particle or cell imaging area; and a channel or tube connected to the target cell or particle outlet and to a first end of a dispenser channel. The one or more sample inlets can be connected to an inlet channel or tube, which can be controlled by a valve. The channel or tube connected to the target cell or particle outlet and to a first end of a dispenser channel can be controlled by a valve. The waste outlet can be connected to waste tube, which can be controlled by a valve. A dispenser channel can comprise the first end and a second end comprising an outlet configured to form a droplet comprising a target cell or target particle and output the droplet. Two air channels can be disposed along a portion of the dispenser channel adjacent to the dispenser channel as describe above. The two air channels can be configured to obtain a flow of pressurized air at an air channel inlet common to the two air channels and to output the flow of pressurized air to an air channel outlet for each of the two air channels. The output of the flow of pressurized air at each air channel outlet can be configured to cause a breakup and removal of the droplet from the outlet of the dispenser channel. In an aspect 1, 2, 3, 4, or more air channels are present. In an aspect, the dispenser can include at least two sheath inlet channels. Each sheath inlet channel can include an inlet configured to obtain a portion of the sample fluid and an outlet outputting the portion of the fluid into the dispenser channel. The at least two sheath inlet channels can be configured to focus the fluid containing the particles or cells to a specified width. In some instances, the specified width of the fluid containing the particle or cell in the dispenser channel is controlled by adjusting a flow rate of the portions of fluid flowing in each of the at least two sheath inlet channels.

The system can further comprise an imaging system comprising at least one light source configured to emit a light onto the cell or particle imaging area to highlight a holographic, fluorescent, bright field, or phase contrast pattern of a cell or particle in the cell or particle imaging area and at least one image sensor (e.g., 1, 2, 3, 4, or more) configured to capture an image of the particle or cell. The image can be an image of the holographic, fluorescent, bright field, or phase contrast pattern of the particles or cells. In an aspect the image comprises only one particle or cell. A mechanism to guide the particle or cell to the waste outlet or to the target particle or target cell outlet based on the image of the particle or cell can also be provided. A controllable switch can be configured to engage a compressor to send pressurized air through the two air channels when a target cell or particle reaches the second end of the dispenser channel. This causes the target particle or cell to release from the dispenser channel at a desired volume and containing one particle or cell. The target particle or cell can be delivered to a container below the dispenser channel outlet. The container can contain one vessel or several vessels. The container can be present on an x-y stage and can be moved so that each droplet is delivered to a sperate vessel (e.g., individual microtiter plate wells). Alternatively, the cell dispenser can be present on a x-y translation stage and can be moved to dispense the target particle or cell to the sample collector or to the waste collector.

The controllable switch can further be configured to engage a vacuum to extract excess liquid where a droplet at the second end of the dispenser channel does not contain a target particle or cell.

Bidirectional pressurized air-assisted particle dispensing systems can be used to dispense a droplet with a target particle or cell. A fluid sample can be applied to the one or more sample inlets of the system. A light source, e.g., a laser light source, can be directed to the cell or particle imaging area to highlight a holographic pattern, a fluorescent, bright field, or phase contrast pattern of the particle or cell in the cell or particle imaging area. An image sensor can capture an image of the holographic pattern, the fluorescent, bright field, or phase contrast pattern of the particle or cell in the cell or particle imaging area. A computing node can process the image to determine a particle or cell type based on the holographic, fluorescent, bright field, or phase contrast pattern of the particle or cell depicted in the image.

Responsive to determining that the particle or cell type is a target particle or cell type, an air compressor connected to the air channel inlet common to the two air channels can direct a flow of pressurized air to the air channel outlet for each of the two channels to cause breakup and removal of a droplet including the target particle or cell from the outlet of the dispenser channel. When no cell or particle is present in the cell or particle imaging area, a vacuum pump connected to the air channel inlet common to the two air channels can vacuum excess liquid back into the two air channels and away from the outlet of the dispenser channel. Where a non-target cell or non-target particle is identified in the cell or particle imaging area, the non-target cell or non-target particle can be moved to the waste outlet.

### Processes

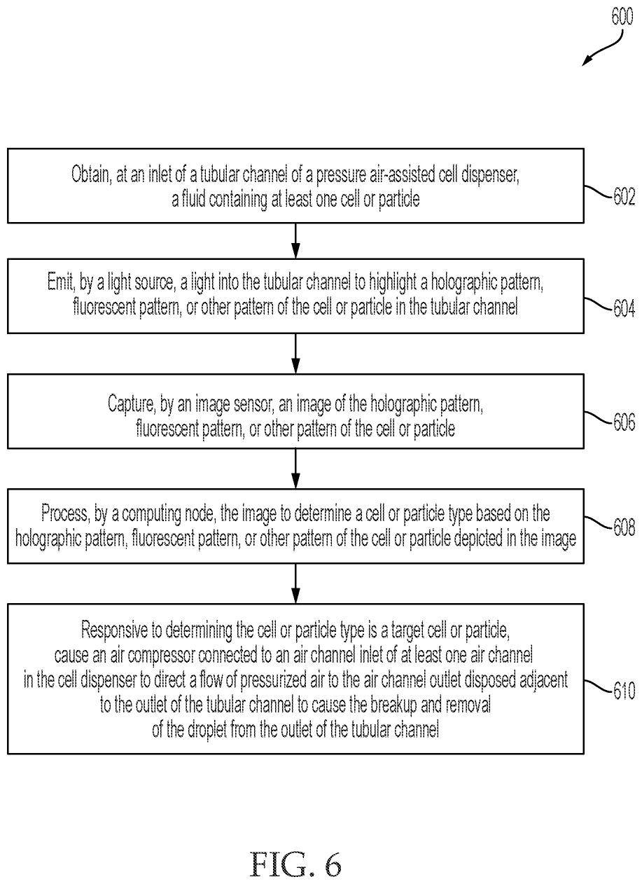

FIG. 6 is a flow process of an example method 600 for dispensing a particle or cell using any pressurized air-assisted particle or cell dispenser described herein. At 602, the method can include obtaining, at an inlet of a tubular channel or dispensing channel of a pressurized air-assisted particle or cell dispenser, a fluid containing at least one particle or cell. The fluid containing the at least one particle or cell can be configured to travel along the tubular channel or dispensing channel to an outlet. The fluid can include a biological fluid, such as a stream of blood or another liquid, such as a saline solution containing one or more particle or cells.

In some instances, the tubular channel or dispensing channel comprises first diameter that is tapered to a second diameter at the outlet, wherein the first diameter is larger than the second diameter. In some instances, the at least one air channel comprises two symmetrical channels disposed on multiple sides of the tubular channels, and each of the two symmetrical channels comprise an outlet that are disposed adjacent to the outlet of the tubular channel or dispensing channel.

In some instances, the method can include directing a portion of the fluid to each of two sheath inlet channels to focus the fluid containing the particle or cell to a specified width within the channel such as the center of the channel or towards a wall of the channel. That is, the particle or cell can be guided to a specific flow path with the channel. Each sheath inlet channel can include an inlet configured to obtain a portion of the fluid and an outlet outputting the portion of the fluid in the tubular channel. In some instances, the method can also include controlling the specified width of the fluid by adjusting a flow rate of the portions of fluid flowing in each of the at least two sheath inlet channels.

In some instances, the tubular channel is part of a particle or cell focusing chip. The at least one air channel can be part of a pneumatic chip. Furthermore, the particle or cell focusing chip can be disposed within the pneumatic chip, and a sheath inlet can be disposed in the particle or cell focusing chip.

At 604, the method can include emitting, by a laser light source, a laser light into the tubular channel or particle or cell imaging area to highlight a holographic, fluorescent, bright field, or phase contrast pattern of the particle or cell in the tubular channel. At 606, the method can include capturing, by an image sensor, an image of the holographic pattern of the particle or cell. In other embodiments, a light source can be used to highlight a fluorescence pattern other suitable pattern (e.g., bright field or phase contrast) of a target particle or cell in the tubular channel or imaging area.

At 608, the method can include processing, by a computing node, the image to determine a particle or cell type based on the holographic pattern of the particle or cell depicted in the image. In other embodiments, the image to determine a target particle or cell type can be based on a fluorescence pattern or other suitable pattern (e.g., bright field, or phase contrast) of a target particle or cell in the tubular channel.

At 610, the method can include responsive to determining that a target particle or cell type (e.g., a circulating tumor cell (CTC)) is present, causing an air compressor connected to an air channel inlet of at least one air channel in the a pressurized air-assisted particle or cell dispenser to direct a flow of pressurized air to the air channel outlet disposed adjacent to the outlet of the tubular channel or dispenser channel to cause the breakup and removal of the droplet from the outlet of the tubular channel.

In a first example embodiment, a pressurized air-assisted particle or cell dispenser is provided. The dispenser can include a tubular channel comprising a first end and a second end. The first end can include an inlet to obtain a fluid containing a particle or cell. The second end can include an outlet configured to form a droplet comprising the particle or cell and output the droplet.

In some instances, the tubular channel or dispenser channel comprises first diameter (e.g., between 50 μm to 1 mm (e.g., about 50, 100, 200, 300, 400, 500, 600, 700, 800, 900, 1,000 μm or more) that is tapered to a second diameter (e.g., about 5 μm, 10, 50, 100, 150, 200, 250, 300, 350, 400, 450, 500 μm or more) at the outlet, wherein the first diameter is larger than the second diameter.

The dispenser can also include at least one air channel disposed along a portion of a pressurized air-assisted particle or cell dispenser adjacent to the tubular channel. The at least one air channel can include an air channel inlet configured to obtain a flow of pressurized air and at least one air channel outlet adjacent to the outlet of the tubular channel. The output of the flow of pressurized air at the air channel outlet can be configured to cause a breakup and removal of the droplet from the outlet of the tubular channel or dispenser channel.

In some instances, the at least one air channel (e.g., about 1, 2, 3, 4, or more air channels) comprises two symmetrical channels disposed on multiple sides of the tubular channels. Each of the two symmetrical channels can include an outlet that are disposed adjacent to the outlet of the tubular channel.

In some instances, the dispenser can include at least two sheath inlet channels. Each sheath inlet channel can include an inlet configured to obtain a portion of the fluid and an outlet outputting the portion of the fluid in the tubular channel. The at least two sheath inlet channels can be configured to focus the fluid containing the particle or cell to a specified width. In some instances, the specified width of the fluid containing the particle or cell in the tubular channel is controlled by adjusting a flow rate of the portions of fluid flowing in each of the at least two sheath inlet channels.

In some instances, the tubular channel is part of a particle or cell focusing chip. The at least one air channel can be part of a pneumatic chip, and the particle or cell focusing chip can be disposed within the pneumatic chip. In some instances, a sheath inlet is disposed in the particle or cell focusing chip.

In some instances, the dispenser can include a holographic imaging system, a fluorescent imaging system, a bright field imaging system, or a phase contrast imaging system, or a combination thereof. The holographic imaging or fluorescent system can include a laser light source configured to emit a laser light into the tubular channel to highlight a holographic pattern, fluorescent, bright field, or phase contrast pattern of the particle or cell in the tubular channel and at least one image sensor configured to capture an image of the holographic, fluorescent, bright field, or phase contrast pattern of the particle or cell.

In some instances, the dispenser can include a computing node including a processor and a memory, with the memory including instructions that, when executed by the processor, cause the processor to perform a series of steps. The steps can include obtaining the image of the holographic, fluorescent, bright field, or phase contrast pattern of the particle or cell, processing the image to determine a particle or cell type based on the holographic, fluorescent, bright field, or phase contrast pattern of the particle or cell depicted in the image, and responsive to determining that the particle or cell type is a target particle or cell (e.g., a circulating tumor cell (CTC)), causing an air compressor connected to the air channel inlet of the at least one air channel to direct the flow of pressurized air to the air channel outlet to cause the breakup and removal of the droplet from the outlet of the tubular channel or dispenser channel.

In another example embodiment, a system is described. The system can include a single particle or cell dispenser that includes: a tubular channel or dispenser channel comprising a first end and a second end, with the first end comprising an inlet to obtain a fluid containing a particle or cell, and the second end comprising an outlet configured to form a droplet comprising the particle or cell and output the droplet. The dispenser can also include 1, 2, 3, 4 or more air channels disposed along a portion of the pressurized air-assisted particle or cell dispenser adjacent to the tubular channel. In an aspect, two air channels can be configured to obtain a flow of pressurized air at an air channel inlet common to the two air channels and output the flow of pressurized air an air channel outlet for each of the two air channels. The output of the flow of pressurized air at each air channel outlet can be configured to cause a breakup and removal of the droplet from the outlet of the tubular channel.

The system can also include a holographic imaging system and/or a fluorescent imaging system comprising a laser light source configured to emit a laser light into the tubular channel to highlight a holographic pattern or a fluorescent pattern of the particle or cell in the tubular channel and at least one image sensor configured to capture an image of the holographic pattern or fluorescent pattern of the particle or cell.

In some instances, the dispenser can further include at least two sheath inlet channels (e.g., 2, 3, 4, or more), with each sheath inlet channel comprising an inlet configured to obtain a portion of the fluid and an outlet outputting the portion of the fluid in the tubular channel. The at least two sheath inlet channels can be configured to focus the fluid containing the particle or cell to a specified width, wherein the specified width of the fluid containing the particle or cell in the tubular channel is controlled by adjusting a flow rate of the portions of fluid flowing in each of the at least two sheath inlet channels.

In some instances, the tubular channel is part of a particle or cell focusing chip, and wherein the at least one air channel is part of a pneumatic chip, where the particle or cell focusing chip is disposed within the pneumatic chip. In some instances, a sheath inlet can be disposed in the particle or cell focusing chip.

In some instances, the system can include a computing node including a processor and a memory, the memory including instructions that, when executed by the processor, cause the processor to perform a series of steps. The steps can include obtaining the image of the holographic pattern of the particle or cell, processing the image to determine a particle or cell type based on the holographic pattern of the particle or cell depicted in the image, and responsive to determining that the particle or cell type is a targeted particle or cell type, causing an air compressor connected to the air channel inlet of the at least one air channel to direct the flow of pressurized air to the air channel outlet to cause the breakup and removal of the droplet from the outlet of the tubular channel.

### Single Cell Isolating Device

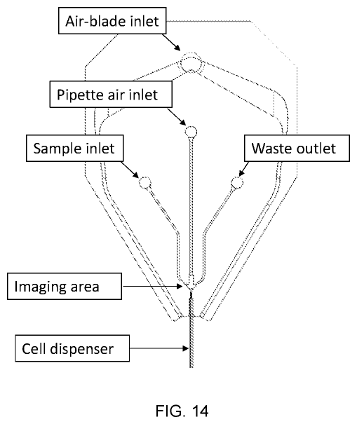

Another aspect provides a single cell isolating device. See FIG. 14. A single cell isolating device can comprise an imaging module as described herein. An imaging module can comprise one more (e.g., 1, 2, 3, 4, or more) cameras or sensors for capturing imaging modalities DIH, fluorescence, bright field, phase contrast, etc. This can include sensors such as photomultiplier tubes, photodiodes, and other sensors. Such sensors can be used to improve the sensitivity and/or sampling rate of the measurements.

A single cell isolating device can include a cell dispensing head comprising a pneumatic microfluidic device, air channels, and a pipette line. A pneumatic microfluidic device comprises two independent air inlets. One inlet is used for 1, 2, 3, 4, or more air channels, which can push and chop the dispensed droplet to a designated size. That is, the air flow can be calibrated based on droplet size generation as described above. Another air inlet is a pipette air inlet connected to a pipette channel, which can push the sample toward the dispenser. The air channels can be as substantially as described above. That is, 1, 2, 3, 4 or more compressed air lumens can be used. Where two air channels are used, they can be located symmetrically on two sides of the dispenser channel and oriented in a shallow angle with respect to the dispenser channel. The air channels can run parallel to the dispenser channel and then angle towards the dispenser outlet. See, e.g., FIG. 3.

A single cell dispenser of FIG. 14 can comprise a microfluidic chip that has one or more inlets for introducing the sample and two outlets for directing the flow. One outlet is connected to the waste collector, and the other is connected to the dispenser to deposit the target cell. That is, a waste channel has a waste outlet and is connected to imaging area. The sample enters through the sample inlet and goes into a sample channel connected to the imaging area. The sample outlet and the waste outlet can be connected to a vacuum source. The vacuum pressure, which is controlled, draws the fluid at the same flow rate with the inlet and directs it to the waste line. The pipette line has a pipette outlet and is connected on its other end to the imaging area. The pipette line is pressurized (connected to a vacuum or compressor) to prevent backflow. The pressure in this the pipette channel is monitored by a barometer, and a proportional integral derivative (PID) control system sets the pressure. When a target cell or particle is detected inside the imaging area (as described in aspects above), the pumps connected to the sample inlet and waste channel are deactivated to retain the particle or cell within the imaging area. Subsequently, the pipette channel increases the air pressure to propel the cell into the dispenser. The pipette channel described for the single cell dispenser can be used in any embodiment described herein.

A microfluidic device can further comprise at least two sheath inlet channels, with each sheath inlet channel comprising an inlet configured to obtain a portion of the fluid and an outlet outputting the portion of the fluid into the sample channel, wherein the at least two sheath inlet channels are configured to focus the fluid containing the particle or cell to a specified width within the sample channel, wherein the specified width within the channel of the fluid containing the particle or cell in the channel is controlled by adjusting a flow rate of the portions of fluid flowing in each of the at least two sheath inlet channels.

A light source as described herein can also be part of the single cell dispensing device. A light source for imaging can be, for example, white light, laser light, LED, xenon arc lamps, tungsten lamps, halogen lamps, fluorescent light, UV light, etc. The wavelengths of the light provided can range between, for example, 350 nanometers (nm) and 800 nm (e.g., 350, 400, 450, 500, 550, 600, 650, 700, 750, or 800 nm).

### Computing System Overview

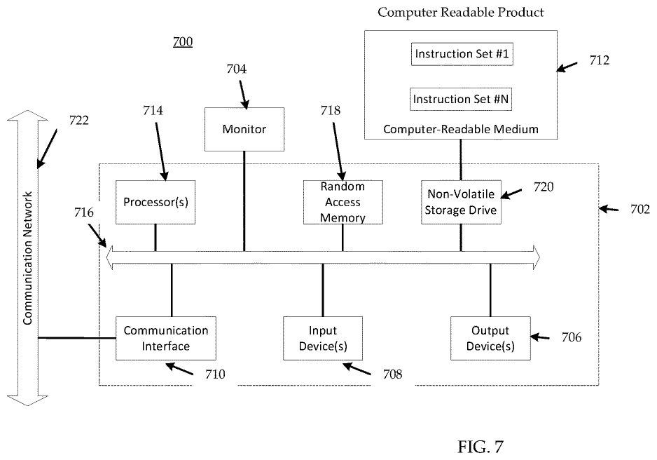

FIG. 7 is a block diagram of a special-purpose computer system 700 according to an embodiment. For example, system 700 can be deployed as part of a computing node as described herein. The methods and processes described herein may similarly be implemented by tangible, non-transitory computer readable storage mediums and/or computer-program products that direct a computer system to perform the actions of the methods and processes described herein. Each such computer-program product can comprise sets of instructions (e.g., codes) embodied on a computer-readable medium that directs the processor of a computer system to perform corresponding operations. The instructions can be configured to run in sequential order, or in parallel (such as under different processing threads), or in a combination thereof.

Special-purpose computer system 700 comprises a computer 702, a monitor 704 coupled to computer 702, one or more additional user output devices 706 (optional) coupled to computer 702, one or more user input devices 708 (e.g., keyboard, mouse, track ball, touch screen) coupled to computer 702, an optional communications interface 710 coupled to computer 702, and a computer-program product including a tangible computer-readable storage medium 712 in or accessible to computer 702. Instructions stored on computer-readable storage medium 712 can direct system 700 to perform the methods and processes described herein. Computer 702 can include one or more processors 714 that communicate with a number of peripheral devices via a bus subsystem 716. These peripheral devices can include user output device(s) 706, user input device(s) 708, communications interface 710, and a storage subsystem, such as random access memory (RAM) 718 and non-volatile storage drive 720 (e.g., disk drive, optical drive, solid state drive), which are forms of tangible computer-readable memory.

Computer-readable medium 712 can be loaded into random access memory 718, stored in non-volatile storage drive 720, or otherwise accessible to one or more components of computer 702. Each processor 714 can comprise a microprocessor, such as a microprocessor from Intel® or Advanced Micro Devices, Inc.®, or the like. To support computer-readable medium 712, the computer 702 runs an operating system that handles the communications between computer-readable medium 712 and the above-noted components, as well as the communications between the above-noted components in support of the computer-readable medium 712. Exemplary operating systems include Windows® or the like from Microsoft Corporation, Solaris® from Sun Microsystems, LINUX, UNIX, and the like. In many embodiments and as described herein, the computer-program product can be an apparatus (e.g., a hard drive including case, read/write head, etc., a computer disc including case, a memory card including connector, case, etc.) that includes a computer-readable medium (e.g., a disk, a memory chip, etc.). In other embodiments, a computer-program product can comprise the instruction sets, or code modules, themselves, and be embodied on a computer-readable medium.

User input devices 708 include all possible types of devices and mechanisms to input information to computer system 702. These can include a keyboard, a keypad, a mouse, a scanner, a digital drawing pad, a touch screen incorporated into the display, audio input devices such as voice recognition systems, microphones, and other types of input devices. In various embodiments, user input devices 708 are typically embodied as a computer mouse, a trackball, a track pad, a joystick, wireless remote, a drawing tablet, a voice command system. User input devices 708 typically allow a user to select objects, icons, text and the like that appear on the monitor 704 via a command such as a click of a button or the like. User output devices 706 include all possible types of devices and mechanisms to output information from computer 702. These can include a display (e.g., monitor 704), printers, non-visual displays such as audio output devices, etc.

Communications interface 710 provides an interface to other communication networks and devices and can serve as an interface to receive data from and transmit data to other systems, WANs and/or the Internet, via a wired or wireless communication network 722. In addition, communications interface 710 can include an underwater radio for transmitting and receiving data in an underwater network. Embodiments of communications interface 710 typically include an Ethernet card, a modem (telephone, satellite, cable, ISDN), a (asynchronous) digital subscriber line (DSL) unit, a FireWire® interface, a USB® interface, a wireless network adapter, and the like. For example, communications interface 710 can be coupled to a computer network, to a FireWire® bus, or the like. In other embodiments, communications interface 710 can be physically integrated on the motherboard of computer 702, and/or can be a software program, or the like.

RAM 718 and non-volatile storage drive 720 are examples of tangible computer-readable media configured to store data such as computer-program product embodiments of the present invention, including executable computer code, human-readable code, or the like. Other types of tangible computer-readable media include floppy disks, removable hard disks, optical storage media such as CD-ROMs, DVDs, bar codes, semiconductor memories such as flash memories, read-only-memories (ROMs), battery-backed volatile memories, networked storage devices, and the like. RAM 718 and non-volatile storage drive 720 can be configured to store the basic programming and data constructs that provide the functionality of various embodiments of the present invention, as described above.

Software instruction sets that provide the functionality of the present aspects can be stored in computer-readable medium 712, RAM 718, and/or non-volatile storage drive 720. These instruction sets or code can be executed by the processor(s) 714. Computer-readable medium 712, RAM 718, and/or non-volatile storage drive 720 can also provide a repository to store data and data structures used in accordance with the present invention. RAM 718 and non-volatile storage drive 720 can include a number of memories including a main random access memory (RAM) to store instructions and data during program execution and a read-only memory (ROM) in which fixed instructions are stored. RAM 718 and non-volatile storage drive 720 can include a file storage subsystem providing persistent (non-volatile) storage of program and/or data files. RAM 718 and non-volatile storage drive 720 can also include removable storage systems, such as removable flash memory.

Bus subsystem 716 provides a mechanism to allow the various components and subsystems of computer 702 communicate with each other as intended. Although bus subsystem 716 is shown schematically as a single bus, alternative embodiments of the bus subsystem can utilize multiple busses or communication paths within the computer 702.

For a firmware and/or software implementation, the methodologies can be implemented with modules (e.g., procedures, functions, and so on) that perform the functions described herein. Any machine-readable medium tangibly embodying instructions can be used in implementing the methodologies described herein. For example, software codes can be stored in a memory. Memory can be implemented within the processor or external to the processor. As used herein the term “memory” refers to any type of long term, short term, volatile, nonvolatile, or other storage medium and is not to be limited to any particular type of memory or number of memories, or type of media upon which memory is stored.

Moreover, as disclosed herein, the term “storage medium” can represent one or more memories for storing data, including read only memory (ROM), random access memory (RAM), magnetic RAM, core memory, magnetic disk storage mediums, optical storage mediums, flash memory devices and/or other machine readable mediums for storing information. The term “machine-readable medium” includes, but is not limited to portable or fixed storage devices, optical storage devices, wireless channels, and/or various other storage mediums capable of storing that contain or carry instruction(s) and/or data.

### Conclusion

It will be understood that terms such as “top,” “bottom,” “above,” “below,” and x-direction, y-direction, and z-direction as used herein as terms of convenience that denote the spatial relationships of parts relative to each other rather than to any specific spatial or gravitational orientation. Thus, the terms are intended to encompass an assembly of component parts regardless of whether the assembly is oriented in the particular orientation shown in the drawings and described in the specification, upside down from that orientation, or any other rotational variation.

The compositions and methods described herein are illustrative only, as numerous modifications and variations therein will be apparent to those skilled in the art. The terms used in the specification generally have their ordinary meanings in the art, within the context of the compositions and methods described herein, and in the specific context where each term is used. Some terms have been more specifically defined herein to provide additional guidance to the practitioner regarding the description of the compositions and methods.

As used herein, the term “and/or” includes any and all combinations of one or more of the associated listed items. As used in the description herein and throughout the claims that follow, the meaning of “a”, “an”, and “the” includes plural reference as well as the singular reference unless the context clearly dictates otherwise. The term “about” in association with a numerical value means that the value varies up or down by 5%. For example, for a value of about 100, means 95 to 105 (or any value between 95 and 105).

All patents, patent applications, and other scientific or technical writings referred to anywhere herein are incorporated by reference herein in their entirety. The embodiments illustratively described herein suitably can be practiced in the absence of any element or elements, limitation or limitations that are specifically or not specifically disclosed herein. Thus, for example, in each instance herein any of the terms “comprising,” “consisting essentially of,” and “consisting of” can be replaced with either of the other two terms, while retaining their ordinary meanings. The terms and expressions which have been employed are used as terms of description and not of limitation, and there is no intention that in the use of such terms and expressions of excluding any equivalents of the features shown and described or portions thereof, but it is recognized that various modifications are possible within the scope of the claims.

Thus, it should be understood that although the present methods and compositions have been specifically disclosed by embodiments and optional features, modifications and variations of the concepts herein disclosed can be resorted to by those skilled in the art, and that such modifications and variations are considered to be within the scope of the compositions and methods as defined by the description and the appended claims.

Any single term, single element, single phrase, group of terms, group of phrases, or group of elements described herein can each be specifically excluded from the claims.

Whenever a range is given in the specification, for example, a temperature range, a time range, a composition, or concentration range, all intermediate ranges and subranges, as well as all individual values included in the ranges given are intended to be included in the disclosure. It will be understood that any subranges or individual values in a range or subrange that are included in the description herein can be excluded from the aspects herein. It will be understood that any elements or steps that are included in the description herein can be excluded from the claimed compositions or methods.

In addition, where features or aspects of the compositions and methods are described in terms of Markush groups or other grouping of alternatives, those skilled in the art will recognize that the compositions and methods are also thereby described in terms of any individual member or subgroup of members of the Markush group or other group.

What is claimed is:

1. A pressurized air-assisted dispenser comprising:
a channel comprising a first end and a second end, the first end comprising an inlet to obtain a fluid containing particles or cells, and the second end comprising an outlet configured to form a droplet comprising a single target particle or cell and output the droplet; and
at least one air channel disposed along a portion of the pressurized air-assisted dispenser adjacent to the channel, wherein the at least one air channel comprises an air channel inlet configured to obtain a flow of pressurized air and at least one air channel outlet adjacent to the outlet of the channel, wherein the output of the flow of pressurized air at the air channel outlet is configured to cause a breakup and removal of the droplet from the outlet of the channel.

2. The pressurized air-assisted dispenser of claim 1, wherein the channel comprises first diameter that is tapered to a second diameter at the outlet, wherein the first diameter is larger than the second diameter.

3. The pressurized air-assisted dispenser of claim 1, wherein the at least one air channel comprises two symmetrical channels disposed on multiple sides of the channel, and wherein each of the two symmetrical channels comprise an outlet that are disposed adjacent to the outlet of the channel.

4. The pressurized air-assisted dispenser of claim 1, further comprising:
at least one sheath inlet channel, the sheath inlet channel comprising an inlet configured to obtain a portion or portions of the fluid and an outlet outputting the portion of the fluid into the channel, wherein the at least one sheath inlet channels is configured to focus the fluid containing the particle or cell to a specified width within the channel, and wherein fluid in the sheath inlet channel include one or more molecular barcodes for tagging the particle or cell.

5. The pressurized air-assisted dispenser of claim 4, wherein fluid in the sheath inlet channel includes one or more molecular barcodes for tagging the particle or cell.

6. The pressurized air-assisted dispenser of claim 5, wherein at least one sheath inlet channel comprises two sheath inlet channels, wherein the specified width within the channel of the fluid containing the particles or cells in the channel is controlled by adjusting a flow rate of the portions of fluid flowing in each of the at least two sheath inlet channels.

7. The pressurized air-assisted dispenser of claim 1, wherein the channel is part of a particle or cell focusing chip, and wherein the at least one air channel is part of a pneumatic chip, where the particle or cell focusing chip is disposed within the pneumatic chip.

8. The pressurized air-assisted dispenser of claim 7, further comprising at least one sheath inlet disposed in the particle or cell focusing chip.

9. The pressurized air-assisted dispenser of claim 1, further comprising:
an imaging system comprising:
a light source configured to emit a light into the channel to highlight a holographic pattern and/or a fluorescent pattern of the particle or cell in the channel; and
at least one image sensor configured to capture an image of the holographic pattern and/or the fluorescent pattern of the particle or cell.

10. The pressurized air-assisted dispenser of claim 9, further comprising:
a computing node including a processor and a memory, the memory including instructions that, when executed by the processor, cause the processor to:
obtain the image of the holographic pattern and/or the fluorescent pattern of the particle or cell;
process the image to determine a particle or cell type based on the holographic pattern and/or fluorescent pattern of the particle or cell depicted in the image; and
responsive to determining that the particle or cell type is a target particle or cell type, causing an air compressor connected to the air channel inlet of the at least one air channel to direct the flow of pressurized air to the air channel outlet to cause the breakup and removal of the droplet from the outlet of the channel, wherein the breakup and removal of the droplet is coordinated with movement of a sample collector or a waste collector moved via a translation stage.

11. A method comprising:
obtaining, at an inlet of a channel of a pressurized air-assisted dispenser, a fluid containing at least one particle or cell, wherein the fluid containing the at least one particle or cell is configured to travel along the channel to an outlet;
emitting, by a laser light source, a laser light into the channel to highlight a holographic pattern and/or a fluorescent pattern of the particle or cell in the channel;
capturing, by an image sensor, an image of the holographic pattern and/or the fluorescent pattern of the particle or cell;
processing, by a computing node, the image to determine a particle or cell type based on the holographic pattern of the particle or cell depicted in the image; and
responsive to determining that the particle or cell type is a target particle or cell type, causing an air compressor connected to an air channel inlet of at least one air channel in the pressurized air-assisted dispenser to direct a flow of pressurized air to the air channel outlet disposed adjacent to the outlet of the channel to cause breakup and removal of a droplet including the target particle or cell from the outlet of the channel.

12. The method of claim 11, wherein the channel comprises first diameter that is tapered to a second diameter at the outlet, wherein the first diameter is larger than the second diameter.

13. The method of claim 11, wherein the at least one air channel comprises two symmetrical air channels disposed on multiple sides of the channel, and wherein each of the two symmetrical channels comprise an outlet that are disposed adjacent to the outlet of the channel.

14. The method of claim 11, further comprising:
directing to at least two sheath inlet channels, a portion of the fluid to focus the fluid containing the particle or cell to a specified width within the channel, wherein each sheath inlet channel comprises an inlet configured to obtain a portion of the fluid and an outlet outputting the portion of the fluid into the channel.

15. The method of claim 14, further comprising:
controlling the specified width within the channel of the fluid by adjusting a flow rate of the portions of fluid flowing in each of the at least two sheath inlet channels.

16. The method of claim 11, wherein the channel is part of a particle or cell focusing chip, and wherein the at least one air channel is part of a pneumatic chip, where the particle or cell focusing chip is disposed within the pneumatic chip, and wherein a sheath inlet is disposed in the particle or cell focusing chip.

17. The method of claim 11, wherein the fluid containing at least one particle or cell includes oil droplets encapsulating the at least one particle or cell.

18. A system comprising:
a single particle or cell dispenser that includes:
a channel comprising a first end and a second end, the first end comprising an inlet to obtain a fluid containing target particle or cells, and the second end comprising an outlet configured to form a droplet comprising a single target particle or cell and output the droplet; and
two air channels disposed along a portion of the single particle or cell dispenser adjacent to the channel, wherein the two air channels are configured to obtain a flow of pressurized air at an air channel inlet common to the two air channels and output the flow of pressurized air an air channel outlet for each of the two air channels, wherein the output of the flow of pressurized air at each air channel outlet is configured to cause a breakup and removal of the droplet from the outlet of the channel; and
an imaging system comprising:
a laser light source configured to emit a laser light into the channel to highlight a holographic and/or fluorescent pattern of the particle or cells in the channel; and
at least one image sensor configured to capture an image of the holographic and/or fluorescent pattern of the particle or cells.

19. The system of claim 18, wherein the single particle or cell dispenser further comprises:
at least two sheath inlet channels, with each sheath inlet channel comprising an inlet configured to obtain a portion of the fluid and an outlet outputting the portion of the fluid in the channel, wherein the at least two sheath inlet channels are configured to focus the fluid containing the particle or cell to a specified width within the channel, wherein the specified width within the channel of the fluid containing the particle or cell in the channel is controlled by adjusting a flow rate of the portions of fluid flowing in each of the at least two sheath inlet channels.

20. The system of claim 18, wherein the channel is part of a particle or cell focusing chip, and wherein the two air channels are part of a pneumatic chip, where the particle or cell focusing chip is disposed within the pneumatic chip.

21. The system of claim 20, wherein the single particle or cell dispenser further comprises a sheath inlet disposed in the particle or cell focusing chip.

22. The system of claim 18, further comprising:
a computing node including a processor and a memory, the memory including instructions that, when executed by the processor, cause the processor to:
obtain the image of the holographic pattern and/or the fluorescent pattern of the particle or cell;
process the image to determine a particle or cell type based on the holographic pattern and/or the fluorescent pattern of the particle or cell depicted in the image; and
responsive to determining that the particle or cell type is a target particle or cell, causing an air compressor connected to the air channel inlet of the two air channels direct the flow of pressurized air to each air channel outlet to cause the breakup and removal of the droplet from the outlet of the channel.

23. The system of claim 22, wherein the cell is circulating tumor cell or an immune cell.

23. A pressurized air-assisted particle dispensing system comprising:
a microfluidic device comprising one or more sample inlets for introducing a fluid sample, a waste outlet, a target particle or cell outlet, and a particle or cell imaging area, and a channel or tube connected to the target cell or particle outlet and to a first end of a dispenser channel;
the dispenser channel comprising a first end and a second end comprising an outlet configured to form a droplet comprising a target cell or target particle and output the droplet;
two air channels disposed along a portion of the dispenser channel adjacent to the dispenser channel, wherein the two air channels are configured to obtain a flow of pressurized air at an air channel inlet common to the two air channels and output the flow of pressurized air to an air channel outlet for each of the two air channels, wherein the output of the flow of pressurized air at each air channel outlet is configured to cause a breakup and removal of the droplet from the outlet of the dispenser channel.

24. The pressurized air-assisted particle dispensing system of claim 23, further comprising an imaging system comprising:
at least one light source configured to emit a light onto the cell or particle imaging area to highlight a holographic and/or fluorescent pattern of a cell or particle in the cell or particle imaging area; and
at least one image sensor configured to capture an image of the particle or cell.

25. The pressurized air-assisted particle dispensing system of claim 23, further comprising a mechanism to guide the particle or cell to the waste outlet or to the target particle or target cell outlet based on the image of the particle or cell.

26. The pressurized air-assisted particle dispensing system of claim 23, further comprising a controllable switch configured to engage a compressor to send pressurized air through the two air channels when a target cell or particle reaches the second end of the dispenser channel.

27. The pressurized air-assisted particle dispensing system of claim 26, wherein the controllable switch is further configured to engage a vacuum to extract excess liquid where a droplet at the second end of the dispenser channel does not contain a target particle or cell.

28. The pressurized air-assisted particle dispensing system of claim 23, wherein the particles are cells.

29. The pressurized air-assisted particle dispensing system of claim 24, wherein the image is an image of the holographic and/or fluorescent pattern of the cells.

30. The pressurized air-assisted particle dispensing system of claim 23, further comprising a waste tube connected to the waste outlet.

31. The pressurized air-assisted particle dispensing system of claim 30, wherein the waste tube connected to the waste outlet is controlled by a valve.

32. The pressurized air-assisted particle dispensing system of claim 23, wherein the channel or tube connected to the target particle or cell outlet and to a first end of a dispenser channel is controlled by a valve.

33. The pressurized air-assisted particle dispensing system of claim 23, further comprising an inlet channel or tube connected to the one or more sample inlets.

34. The pressurized air-assisted particle dispensing system of claim 33, wherein the inlet channel or tube is controlled by a valve.

35. A method of dispensing a droplet with a particle or cell comprising applying a fluid sample to the one or more sample inlets of the pressurized air-assisted particle dispensing system of claim 24,
emitting, by a laser light source, a laser light onto the cell or particle imaging area to highlight a holographic pattern and/or a fluorescent pattern of the particle or cell in the cell or particle imaging area;
capturing, by an image sensor, an image of the holographic pattern and/or the fluorescent pattern of the particle or cell;
processing, by a computing node, the image to determine a particle or cell type based on the holographic and/or fluorescent pattern of the particle or cell depicted in the image; and
responsive to determining that the particle or cell type is a target particle or cell type, causing an air compressor connected to the air channel inlet common to the two air channels to direct a flow of pressurized air to the air channel outlet for each of the two channels to cause breakup and removal of a droplet including the target particle or cell from the outlet of the dispenser channel.

36. The method of claim 35, further comprising, when no cell or particle is present in the cell or particle imaging area, causing a vacuum pump connected to the air channel inlet common to the two air channels to vacuum excess liquid back into the two air channels and away from the outlet of the dispenser channel.

37. The method of claim 35, further comprising, responsive to determining that the particle or cell type is a non-target particle or cell type, causing the particle or cell to the waste outlet.

38. A single cell dispensing system comprising:
a microfluidic device comprising one or more sample inlets for introducing a fluid sample, a waste outlet, a dispenser channel having a target particle or cell outlet, a particle or cell imaging area, and a pipette air inlet;
the target particle or cell outlet is configured to form a droplet comprising a target cell or target particle and output the droplet;
two air channels disposed along a portion adjacent to the dispenser channel, wherein the two air channels are configured to obtain a flow of pressurized air at an air channel inlet common to the two air channels and output the flow of pressurized air to an air channel outlet for each of the two air channels, wherein the output of the flow of pressurized air at each air channel outlet is configured to cause a breakup and removal of the droplet from the outlet of the dispenser channel.

39. The single cell dispensing system of claim 38, wherein the pipette air inlet is configured to be pressurized to prevent backflow and upon detection of a target particle or cell in the imaging area can be pressurized to push a target particle or cell to the target particle or cell outlet.

40. The single cell dispensing system of claim 38, wherein the sample inlet and waste outlet are connected to a vacuum source.

41. The single cell dispensing system of claim 38, further comprising an imaging or sensing system comprising:
at least one light source configured to emit a light onto the cell or particle imaging area to highlight a holographic and/or fluorescent pattern of a cell or particle in the cell or particle imaging area; and
at least one sensor configured to capture an image or signal of the particle or cell.

42. The single cell dispensing system of claim 38, further comprising a controllable switch configured to engage a compressor to send pressurized air through the two air channels when a target cell or particle reaches the particle or cell outlet.

43. The single cell dispensing system of claim 38, wherein the pipette air inlet is pressurized.

44. The single cell dispensing system of claim 38, further comprising a controllable switch configured to engage a vacuum in the waste inlet and sample inlet until a target cell or particle is detected in the particle or cell imaging area, such that the vacuum is turned off and the pipette inlet becomes pressurized thereby moving the target particle or cell to the particle or cell outlet.

45. A method of dispensing a droplet with a particle or cell comprising applying a fluid sample to the one or more sample inlets of the single cell dispensing system of claim 38,
emitting, by a laser light source, a laser light onto the cell or particle imaging area to highlight a holographic pattern and/or a fluorescent pattern of the particle or cell in the cell or particle imaging area;
capturing, by an image sensor, an image of the holographic pattern and/or the fluorescent pattern of the particle or cell;
processing, by a computing node, the image to determine a particle or cell type based on the holographic and/or fluorescent pattern of the particle or cell depicted in the image; and responsive to determining that the particle or cell type is a target particle or cell type turning off the vacuum in the waste inlet and sample inlet.

46. The method of claim 45, the processing further comprising responsive to determining that the particle or cell type is a target particle or cell type, causing an air compressor connected to the air channel inlet common to the two air channels to direct a flow of pressurized air to the air channel outlet for each of the two channels to cause breakup and removal of a droplet including the target particle or cell from the outlet of the dispenser channel.
